(19) United States
(12) Patent Application Publication
(10) Pub. No.: US 2024/0376028 A1
(43) Pub. Date: Nov. 14, 2024

(54) METHOD FOR PRODUCING trans-1,2-DIFLUOROETHYLENE (HFO-1132E)

(71) Applicant: Honeywell International Inc., Charlotte, NC (US)

(72) Inventors: Haiyou Wang, Amherst, NY (US); Alexey Kruglov, Belmont, WA (US); Yian Zhai, Lockport, NY (US); Dimitrios Papanastasiou, Buffalo, NY (US); Sudharsanam Ramanathan, Buffalo, NY (US); Christophe Roger, Buffalo, NY (US); Hsien-cheng YU, Charlotte, NC (US); Akbar Mahdavi-Shakib, Buffalo, NY (US)

(21) Appl. No.: 18/658,300

(22) Filed: May 8, 2024

Related U.S. Application Data
(60) Provisional application No. 63/465,142, filed on May 9, 2023.
(60) Provisional application No. 63/534,001, filed on Aug. 22, 2023.

(51) Int. Cl. C07C 17/23; C07C 17/25

(52) U.S. Cl. CPC C07C 17/23; C07C 17/25; C07B 2200/09

(57) ABSTRACT

Production of HFO-1132 and, in particular, HFO-1132E, may be produced from 1,1,2-trichloro-1,2,2-trifluoroethane (CFC-113). In a first step, 1,1,2-trifluoroethane (HFC-143) is produced by hydrogenating 1,1,2-trichloro-1,2,2-trifluoroethane (CFC-113) by reaction with hydrogen in the presence of a catalyst to produce 1,1,2-trifluoroethane (HFC-143). The 1,1,2-trifluoroethane (HFC-143) may then be dehydrofluorinated in the presence of a catalyst to produce trans-1,2-difluoroethylene (HFO-1132E) and/or cis-1,2-difluoroethylene (HFO-1132Z). The cis-1,2-difluoroethylene (HFO-1132Z) may then be isomerized to produce trans-1,2-difluoroethylene (HFO-1132E).

### CROSS-REFERENCE TO RELATED APPLICATIONS

This application claims the benefit under 35 U.S.C. 119(e) of U.S. Provisional Patent Application Ser. No. 63/465,142, filed on May 9, 2023, and U.S. Provisional Patent Application Ser. No. 63/534,001, filed on Aug. 22, 2023, the disclosures of which are incorporated by reference herein in their entireties.

### FIELD

The present disclosure is directed to a method for producing trans-1,2-difluoroethylene (HFO-1132E) from 1,1,2-trichloro-1,2,2-trifluoroethane (CFC-113).

### BACKGROUND

1,2-difluoroethylene (HFO-1132) has recently found increased utility for a variety of uses. HFO-1132 may exist as a mixture of two geometric isomers, the E- or trans isomer and the Z- or cis isomer, which may be used separately or together in various proportions. Potential end use applications of HFO-1132 include refrigerants, either used alone or in blends with other components, solvents for organic materials, and as a chemical intermediate in the synthesis of other halogenated hydrocarbon solvents.

Improved methods for the production of HFO-1132 and, in particular, HFO-1132E, are desired.

### SUMMARY

The present disclosure is based on the discovery that HFO-1132 and, in particular, HFO-1132E, may be produced from 1,1,2-trichloro-1,2,2-trifluoroethane (CFC-113) via catalytic reaction steps in which desirable intermediates are produced in controlled amounts while production of undesirable byproducts is minimized.

In a first step, 1,1,2-trifluoroethane (HFC-143) is produced by hydrogenating 1,1,2-trichloro-1,2,2-trifluoroethane (CFC-113) by reaction with hydrogen in the presence of a catalyst to produce 1,1,2-trifluoroethane (HFC-143). The 1,1,2-trifluoroethane (HFC-143) may then be dehydrofluorinated in the presence of a catalyst to produce trans-1,2-difluoroethylene (HFO-1132E) and/or cis-1,2-difluoroethylene (HFO-1132Z). The cis-1,2-difluoroethylene (HFO-1132Z) may then be isomerized to produce trans-1,2-difluoroethylene (HFO-1132E).

In one form thereof, the present disclosure provides A method for producing 1,1,2-trifluoroethane (HFC-143), comprising: hydrogenating 1,1,2-trichloro-1,2,2-trifluoroethane (CFC-113) by reaction with hydrogen in the presence of a catalyst comprising palladium or platinum to produce a product mixture, wherein the product mixture comprises: at least 30 mol % of 1,1,2-trifluoroethane (HFC-143); and 0.1 mol % to 60 mol % of at least one of 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123), based on of the total moles of organic components in the product mixture.

In another form thereof, the present disclosure provides A method for producing trans-1,2-difluoroethylene (HFO-1132E), comprising: dehydrofluorinating 1,1,2-trifluoroethane (HFC-143) in the presence of a catalyst selected from fluorinated alumina, palladium supported on fluorinated alumina, fluorinated magnesium oxide, and nickel supported on fluorinated alumina to produce a reactant mixture comprising trans-1,2-difluoroethylene (HFO-1132E) and/or cis-1,2-difluoroethylene (HFO-1132Z).

### BRIEF DESCRIPTION OF THE DRAWINGS

FIG. 1 is a schematic diagram of the apparatus used in Example 10A for the conversion of 1,1,2-trichloro-1,2,2-trifluoroethane (CFC-113) to 1,1,2-trifluoroethane (HFC-143).

FIG. 2 is a schematic reactor configuration corresponding to Examples 11-54.

### DETAILED DESCRIPTION

### I. Definitions

As used herein, the singular forms “a”, “an” and “the” include plural unless the context clearly dictates otherwise. Moreover, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the disclosure be limited to the specific values recited when defining a range.

As used herein, the phrase “based on total moles of organic components of the composition” refers only to carbon-containing components and does not include or encompass non-carbon-containing components such as hydrogen (H2) or hydrogen chloride (HCl).

As used herein, conversion of a reactant molecule (molecule X) during a reaction may be calculated using the following equation:

% conversion of molecule X=(100−molecule X mole % in the organic components of a product mixture)

As used herein, selectivity to a molecule formed during a reaction (molecule X) may be calculated using the following equation:

% selectivity to molecule X=mole % of molecule X in the organic components of a product mixture/(100−mole % of reactant molecules in the organic components of a product mixture)×100.

### II. Overview

The present disclosure provides a method for producing E-1,2-difluoroethylene (HFO-1132E) from 1,1,2-trichloro-1,2,2-trifluoroethane (CFC-113) according to a three-step process shown below (“Process 1”), which includes the following three steps: (i) hydrogenating 1,1,2-trichloro-1,2,2-trifluoroethane (CFC-113) to produce 1,1,2-trifluoroethane (HFC-143), (ii) dehydrofluorinating 1,1,2-trifluoroethane (HFC-143) to produce a mixture of trans-1,2-difluoroethylene (HFO-1132E) and cis-1,2-difluoroethylene (HFO-1132Z), and (iii) isomerizing cis-1,2-difluoroethylene (HFO-1132Z) to trans-1,2-difluoroethylene (HFO-1132E).

Schematic equations for the three steps of Process 1 are represented below:

### Process 1

CFCl₂—CF₂Cl(CFC-113)+H₂→CFH₂—CF₂H(HFC-143)+HCl (i)

CFH₂—CF₂H→trans-CFH═CHF(HFO-1132E)+cis-CFH═CFH(HFO-1132Z)+HF (ii)

cis-CFH═CFH(HFO-1132Z)→trans-CFH═CHF(HFO-1132E) (iii)

Step (i) may proceed through an intermediate of 1,1,2-trifluoroethene, wherein 1,1,2-trichloro-1,2,2-trifluoroethane (CFC-113) is first hydrogenated to produce the 1,1,2-trifluoroethene as an intermediate, which intermediate is itself then hydrogenated to produce 1,1,2-trifluoroethane (HFC-143).

Further details regarding each of Steps (i), (ii), (iii) are set forth below.

### III. Step (i)

### General Process

The hydrogenation reaction of Step (i) may be carried out in the gas vapor phase in a suitable reactor, for example a tubular reactor made from a material which is resistant to temperature and/or corrosion such as nickel and its alloys, including Hastelloy (for example, Hastelloy C276), Inconel (for example, Inconel 600), Incoloy, and Monel, and the vessels may be lined with fluoropolymers.

The reactor may be first cleaned and flushed with an inert gas such as nitrogen, followed by packing with a catalyst such as those described below. The catalyst may be pretreated within the reactor such as by drying in the manner described further below, followed by metering the reactants into the reactor to initiate the reaction.

The process flow may be in the down or up direction through a bed of the catalyst. Products may be flowed through one or more scrubbers to remove by-products from the reaction, such as hydrogen fluoride (HF) and/or hydrogen chloride (HCl), and the reaction products may be collected by capture in a cooled cylinder, for example.

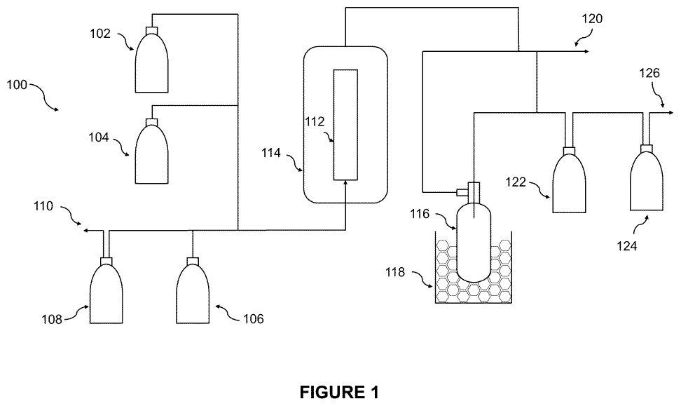

One schematic of a process flow illustrating suitable components for the reaction in step (i) is provided in FIG. 1. Referring to the process flow diagram 100 shown therein, a supply of nitrogen (N₂), which acts as an inert carrier, is provided from cylinder 102 and a supply of hydrogen (H₂) is provided from cylinder 104. The organic feedstock which comprises 1,1,2-trichloro-1,2,2-trifluoroethane (CFC-113), is supplied from cylinder 106. Cylinders 102, 104 and 106 are all connected to KOH scrubber 108 which is coupled to vent 110. The supply of N₂, H₂, and organic feedstock is fed into reactor 112 which is surrounded by box oven 114. The reaction may be monitored by taking samples from outlet 120 and conducting a GC analysis. As the reaction proceeds, the products are fed into holding tank 116 which may be immersed in a bath of dry ice or a dry ice and acetone mixture 118. The dry ice or dry ice and acetone mixture bath is configured to hold the temperature of holding tank 116 at a reduced temperature below ambient, such as about −87° C., for example. Holding tank 116 is coupled to buffer knock-out tank 122 to prevent potential backflow of KOH solution, which is also coupled to KOH scrubber 124. Scrubber 124 has a vent 126 which opens to the atmosphere. The catalyst and process conditions play an important role in the reaction. Specifically, in the hydrogenation Step (i), the catalyst may comprise a metal such as palladium, platinum, iron, cobalt or nickel.

### Catalysts

The catalyst active to catalyze the reaction may be palladium metal, platinum metal, or a combination of palladium metal and platinum metal.

The catalyst may be supported on a suitable support, such as carbon or alumina. The carbon may be activated carbon. The alumina may be alpha alumina, theta alumina, delta alumina, or gamma alumina. The supported catalyst may be produced by impregnation of any of the suitable supports with a solution of a compound of the desired metal constituent. The support may also be in the form of pellets. After the impregnation step, the solvent may be removed using heat or under vacuum resulting in a solid mass which can be further dried and reduced to form active metal catalyst.

The catalyst may be palladium on a carbon support, may be platinum on a carbon support, and/or may be palladium or platinum on an alumina support. In one particularly preferred embodiment, the catalyst is palladium on an alpha alumina support.

The amount of metal loading on the support may be from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, about 5 wt. %, about 10 wt. %, about 20 wt. %, about 30 wt. %, about 40 wt. %, about 50 wt. % or within any range encompassed by two of the foregoing values as endpoints, based on a total weight of the catalyst and support. For supported noble metal catalysts such as Pd or Pt, the metal loading may be from about 0.01 wt. % to about 5 wt. %, preferably from about 0.05 wt. % to about 2 wt. %, and more preferably from about 0.1 wt. % to about 1 wt. %.

When a palladium catalyst is used, the loading of palladium on the support, such as an alpha alumina support, may be from about 0.01 wt. % to about 5 wt. %, preferably from about 0.05 wt. % to about 2 wt. %, and more preferably from about 0.1 wt. % to about 1 wt. %.

The catalyst used in step (i) may have a proper BET (Brunauer, Emmet, and Teller) surface area. In some embodiments, the BET surface area of the catalyst may be as low as about 1 m²/g, about 3 m²/g, about 5 m²/g, about 10 m²/g, about 15 m²/g, about 20 m²/g², about 30 m²/g, about 40 m²/g, about 50 m²/g, about 100 m²/g, about 200 m²/g, or as high as about 250 m²/g, about 300 m²/g, about 400 m²/g, about 500 m²/g, about 600 m²/g, about 700 m²/g m², about 800 m²/g, about 900 m²/g, about 1000 m²/g, about 2000 m²/g, or within any range encompassed by any of the foregoing values as endpoints.

For alumina supported metal catalysts, the BET surface area may be from about 1 m²/g to about 500 m²/g, preferably from about 1 m²/g to about 200 m²/g, more preferably from about 1 m²/g to about 100 m²/g, and most preferably from about 1 m²/g to about 20 m²/g.

When a palladium catalyst is used on an alpha alumina support, the BET surface area may be from about 1 m²/g to about 500 m²/g, preferably from about 1 m²/g to about 200 m²/g, more preferably from about 1 m²/g to about 100 m²/g, and most preferably from about 1 m²/g to about 20 m²/g.

The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

### Catalyst Pretreatment

The catalyst may be pretreated by a variety of methods to improve its performance and effectiveness in the reaction. For example, the catalyst may be dried at elevated temperatures, as low as about 200° C., about 250° C., about 300° C., about 350° C., about 360° C., about 370° C., or as high as about 380° C., about 390° C., about 400° C., about 450° C., about 500° C., about 600° C., about 700° C., or within any range encompassed by two of the foregoing values as endpoints. As part of the catalyst pretreatment, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints such as about 2 hours to about 4 hours, for example.

When a palladium catalyst is used on an alpha alumina support, the catalyst may be dried at a temperature of from about 200° C. to about 700° C., preferably from about 200° C. to about 500° C., most preferably from about 200° C. to about 300° C.

When a palladium catalyst is used on an alpha alumina support, the catalyst may be exposed to an inert gas such as N₂ for from about 1 hour to about 20 hours, preferably from about 1 hour to about 10 hours, most preferably, from about 1 hour to about 3 hours.

### Reaction Conditions

The reaction temperature may be as low as about 100° C., about 125° C., about 150° C., about 200° C., about 250° C. or as high as about 300° C., about 350° C., about 400° C., or within any range encompassed by two of the foregoing values as endpoints, such as from about 100° C. to about 250° C., or from about 150° C. to about 200° C., for example. The temperature may be preferably from about 100° C. to about 300° C., and more preferably from about 150° C. to about 250° C.

When a palladium catalyst is used on an alpha alumina support, the reaction temperature may be from about 100° C. to about 400° C., preferably from about 100° C. to about 300° C., most preferably from about 150° C. to about 250° C.

As demonstrated by the Examples herein, the selectivity towards the desired product 1,1,2-trifluoroethane (HFC-143) may increase with temperature. However, the overall selectivity to 1,1,2-trifluoroethane (HFC-143) and its associated recyclable intermediates such as 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) may decrease with high temperature because of increased formation of undesired by-products such as ethane (HC-170), chloroethane (HCC-160), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)), 1,1,1-trifluoroethane (HFC-143a) etc.

The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be preferably from about 1 second to about 60 seconds.

When a palladium catalyst is used on an alpha alumina support, the contact time may be from about 1 second to about 60 seconds, preferably from about 5 seconds to about 40 seconds, most preferably from about 10 seconds to about 30 seconds.

The pressure may be as little as about 1 psig, about 3 psig, about 5 psig, about 10 psig, about 15 psig, about 20 psig, about 30 psig, about 35 psig or about 40 psig, or as great as about 90 psig, about 100 psig, about 120 psig, about 150 psig, about 200 psig or about 250 psig, about 300 psig, or within any range encompassed by two of the foregoing values as endpoints. For example, the pressure may be preferably from about 10 psig to about 100 psig.

When a palladium catalyst is used on an alpha alumina support, the pressure may be from about 1 psig to about 300 psig, preferably from about 1 psig to about 200 psig, most preferably from about 10 psig to about 100 psig.

The mole ratio of hydrogen to 1,1,2-trichloro-1,2,2-trifluoroethane (CFC-113) may be as little about 2:1, about 3:1, about 4:1, about 5:1, about 5.5:1 or as great as about 6:1, about 6.5:1, about 7.5:1 or about 8:1, about 12:1, about 15:1, or about 20:1, for example, or within any range encompassed by two of the foregoing values as endpoints. The mole ratio of hydrogen to CFC-113 may be preferably from about 3:1 about to 15:1, and more preferably from about 4:1 to about 10:1.

When a palladium catalyst is used on an alpha alumina support, the mole ratio of hydrogen to 1,1,2-trichloro-1,2,2-trifluoroethane (CFC-113) may be from about 2:1 to about 20:1, preferably from about 3:1 about to 15:1, most preferably from about 4:1 to about 10:1.

### Products

The hydrogenation step (step (i)) may produce a product mixture in the reactor comprising of the desired product 1,1,2-trifluoroethane (HFC-143); desired intermediates, such as 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123); and undesirable by products, such as 1,1-difluoroethane (HFC-152a), ethane (HC-170), chloroethane (HCC-160), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)), and 1,1,1-trifluoroethane (HFC-143a).

As demonstrated by the Examples herein, the hydrogenation step may achieve a selectivity to the 1,1,2-trifluoroethane (HFC-143) product of greater than about 20%, greater than about 30%, greater than about 40%, greater than about 50%, greater than about 60%, greater than about 70%, greater than about 80%, greater than about 90%, or within any range encompassed by two of the foregoing values as endpoints.

When a palladium catalyst is used on an alpha alumina support, the hydrogenation step may achieve a selectivity to the 1,1,2-trifluoroethane (HFC-143) product of greater than about 20%, preferably greater than about 30%, most preferably greater than about 40%.

The hydrogenation reaction may also produce several intermediates such as 1-chloro-1,1,2-trifluoroethane (HCFC-133b), 1-chloro-1,2,2-trifluoroethane (HCFC-133), and 1,2-dichloro-1,1,2-trifluoroethane (HCFC-123a), and trifluoroethylene (HFO-1123). These intermediates are recyclable and can be eventually converted to 1,1,2-trifluoroethane (HFC-143).

As demonstrated by the Examples herein, the hydrogenation step may achieve a combined selectivity and/or selectivity to each of 1,1,2-trifluoroethane (HFC-143), 1-chloro-1,1,2-trifluoroethane (HCFC-133b), and 1,2-dichloro-1,1,2-trifluoroethane (HCFC-123a) of greater than about 30%, greater than about 40%, greater than about 50%, greater than about 60%, greater than about 70%, greater than about 80%, greater than about 90%, greater than about 95%, or within any range encompassed by two of the foregoing values as endpoints.

When a palladium catalyst is used on an alpha alumina support, the hydrogenation step may achieve a combined selectivity and/or selectivity to each of 1,1,2-trifluoroethane (HFC-143), 1-chloro-1,1,2-trifluoroethane (HCFC-133b), and 1,2-dichloro-1,1,2-trifluoroethane (HCFC-123a) of greater than about 30%, preferably greater than about 40%, most preferably greater than about 50%.

The hydrogenation reaction may also produce several by-products such as 1,1-difluoroethane (HFC-152a), ethane (HC-170), 1,1,1-trifluoroethane (HFC-143a), 1,1-difluoroethylene (HFO-1132a), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)), and chloroethane (HCC-160) which are the result of hydrodehalogenation side reactions. These by-products are undesirable as they are difficult to recycle or convert to 1,1,2-trifluoroethane (HFC-143). As demonstrated by the Examples herein, the hydrogenation step may achieve a combined selectivity and/or selectivity to each of to 1,1-difluoroethane (HFC-152a), ethane (HC-170), 1,1,1-trifluoroethane (HFC-143a), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)), and chloroethane (HCC-160) of less than about 30%, less than about 25%, less than about 20%, less than about 15%, less than about 10%, less than about 9%, less than about 8%, less than about 7%, less than about 6%, less than about 5%, or within any range encompassed by two of the foregoing values as endpoints.

When a palladium catalyst is used on an alpha alumina support, the hydrogenation step may achieve a combined selectivity and/or selectivity to each of to 1,1-difluoroethane (HFC-152a), ethane (HC-170), 1,1,1-trifluoroethane (HFC-143a)), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)), and chloroethane (HCC-160) of less than about 30%, preferably less than about 25%, most preferably less than about 20%.

As also demonstrated by the Examples, herein, the hydrogenation step may achieve a conversion of 1,1,2-trichloro-1,2,2-trifluoroethane (CFC-113) to 1,1,2-trifluoroethane (HFC-143) of greater than about 10%, greater than about 20%, greater than about 30%, greater than about 40%, greater than about 50% greater than about 60%, greater than about 75%, greater than about 90%, greater than about 95%, greater than about 97% or greater, for example, or within any range encompassed by two of the foregoing values as endpoints.

When a palladium catalyst is used on an alpha alumina support, the hydrogenation step may achieve a conversion of 1,1,2-trichloro-1,2,2-trifluoroethane (CFC-113) to 1,1,2-trifluoroethane (HFC-143) of greater than about 10%, preferably greater than about 20%, most preferably greater than about 30%.

It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrogen treatment at temperatures of from about 100° C. to about 400° C., preferably from about 200° C. to about 350° C. for carbon and alumina supported metal catalysts.

When a palladium catalyst is used on an alpha alumina support, one or more of the following properties may be present. The loading of palladium on the support may be from about 0.01 wt. % to about 5 wt. %, preferably from about 0.05 wt. % to about 2 wt. %, and more preferably from about 0.1 wt. % to about 1 wt. %. The BET surface area may be from about 1 m²/g to about 500 m²/g, preferably from about 1 m²/g to about 200 m²/g, more preferably from about 1 m²/g to about 100 m²/g, and most preferably from about 1 m²/g to about 20 m²/g. The catalyst may be dried at a temperature of from about 200° C. to about 700° C., preferably from about 200° C. to about 500° C., most preferably from about 200° C. to about 300° C. The catalyst may be exposed to an inert gas such as N₂ for from about 1 hour to about 20 hours, preferably from about 1 hour to about 10 hours, most preferably, from about 1 hour to about 3 hours. The reaction temperature may be from about 100° C. to about 400° C., preferably from about 100° C. to about 300° C., most preferably from about 150° C. to about 250° C. The contact time may be from about 1 second to about 60 seconds, preferably from about 5 seconds to about 40 seconds, most preferably from about 10 seconds to about 30 seconds. The pressure may be from about 1 psig to about 300 psig, preferably from about 1 psig to about 200 psig, most preferably from about 10 psig to about 100 psig. The mole ratio of hydrogen to 1,1,2-trichloro-1,2,2-trifluoroethane (CFC-113) may be from about 2:1 to about 20:1, preferably from about 3:1 about to 15:1, most preferably from about 4:1 to about 10:1.

The hydrogenation step may achieve a selectivity to the 1,1,2-trifluoroethane (HFC-143) product of greater than about 20%, preferably greater than about 30%, most preferably greater than about 40%.

The hydrogenation step may achieve a combined selectivity and/or selectivity to each of 1,1,2-trifluoroethane (HFC-143), 1-chloro-1,1,2-trifluoroethane (HCFC-133b), and 1,2-dichloro-1,1,2-trifluoroethane (HCFC-123a) of greater than about 30%, preferably greater than about 40%, most preferably greater than about 50%.

The hydrogenation step may achieve a combined selectivity and/or selectivity to each of to 1,1-difluoroethane (HFC-152a), ethane (HC-170), 1,1,1-trifluoroethane (HFC-143a), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)), and chloroethane (HCC-160) of less than about 30%, preferably less than about 25%, most preferably less than about 20%.

The hydrogenation step may achieve a conversion of 1,1,2-trichloro-1,2,2-trifluoroethane (CFC-113) to 1,1,2-trifluoroethane (HFC-143) of greater than about 10%, preferably greater than about 20%, most preferably greater than about 30%.

In the above process, referring to FIG. 1, the amount of 1,1,2-trifluoroethane (HFC-143) in the product mixture may be at least 30 mol %, at least 50 mol %, or at least 70 mol. %, for example, based on the total moles of organic components of the product mixture.

The amount of 1-chloro-1,1,2-trifluoroethane (HCFC-133b) in the product mixture, if present, may be greater than 0.1 mol %, and yet less than 60 mol %, less than 30 mol %, or less than 10 mol %, for example, based on the total moles of organic components of the product mixture, for example from 0.1 mol % to 60 mol %, from 0.1 mol % to 30 mol %, or from 0.1 mol % to 10 mol %.

The amount of 1-chloro-1,2,2-trifluoroethane (HCFC-133) in the product mixture, if present, may be greater than 0.1 mol %, and yet less than 20 mol %, less than 10 mol %, or less than 5 mol %, for example, based on the total moles of organic components of the product mixture, for example from 0.1 mol % to 20 mol %, from 0.1 mol % to 10 mol %, or from 0.1 mol % to 5 mol %.

The amount of 1,2-dichloro-1,1,2-trifluoroethane (HCFC-123a) in the product mixture, if present, may be greater than 0.1 mol. %, and yet less than 20 mol %, less than 10 mol %, or less than 5 mol %, for example, based on the total moles of organic components in the product mixture for example from 0.1 mol % to 20 mol %, from 0.1 mol % to 10 mol %, or from 0.1 mol % to 5 mol %.

The total amount of 1,1,2-trifluoroethane (HFC-143), 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) in the product mixture may be at least 60 mol %, at least 70 mol %, or at least 90 mol %, for example, based on the total moles of organic components in the product mixture.

The total amount of 1,1-difluoroethane (HFC-152a) in the product mixture, if present, may be greater than 0.1 mol % and less than 10 mol %, less than 5 mol %, or less than 1 mol %, for example, based on the total moles of organic components in the product mixture for example from 0.1 mol % to 10 mol %, from 0.1 mol % to 5 mol %, or from 0.1 mol % to 1 mol %.

The total amount of ethane (HC-170) in the product mixture, if present, may be greater than 0.1 mol % and less than 5 mol %, less than 3 mol %, or less than 1 mol %, for example, based on the total moles of organic components in the product mixture for example from 0.1 mol % to 5 mol %, from 0.1 mol % to 3 mol %, or from 0.1 mol % to 1 mol %.

The total amount of chloroethane (HCC-160) in the product mixture, if present, may be greater than 0.1 mol % and less than 3 mol %, less than 2 mol %, or less than 1 mol %, for example, based on the total moles of organic components in the product mixture for example from 0.1 mol % to 3 mol %, from 0.1 mol % to 2 mol %, or from 0.1 mol % to 1 mol %.

The total amount of chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)) in the product mixture, if present, may be greater than 0.1 mol % and less than 3 mol %, less than 2 mol %, or less than 1 mol %, for example, based on the total moles of organic components in the product mixture.

The total amount of 1,1,1-trifluoroethane (HFC-143a) in the product mixture, if present, may be greater than 0.1 mol % and less than 3 mol %, less than 2 mol %, or less than 1 mol %, for example, based on the total moles of organic components in the product mixture for example from 0.1 mol % to 3 mol %, from 0.1 mol % to 2 mol %, or from 0.1 mol % to 1 mol %.

### IV. Step (ii)

### General Process

The dehydrofluorination reaction of Step (ii) may be carried out in the vapor phase in a suitable reactor, for example a tubular reactor made from a material which is resistant to temperature and/or corrosion such as nickel and its alloys, including Hastelloy (for example, Hastelloy C276), Inconel (for example Inconel 600), Incoloy, and Monel wherein the vessels which may be lined with fluoropolymers.

The reactor may be first cleaned and flushed with an inert gas such as nitrogen, followed by packing with a catalyst such as those described below. The catalyst may be pretreated within the reactor such as by drying in the manner described further below, followed by metering the reactants into the rector to initiate the reaction.

The process flow may be in the down or up direction through a bed of the catalyst. Reactants may be flowed through a scrubber to remove by-products from the reaction, such as hydrogen fluoride (HF) and/or hydrogen chloride (HCl), and the reaction products may be collected by capture in a cooled cylinder, for example.

### Catalysts

The catalyst and process conditions play an important role in the dehydrofluorination reaction.

Suitable catalysts for the dehydrofluorination reaction include metal oxides such as chromium oxide, aluminum oxide, iron oxide, and magnesium oxide. Fluorination treatment of the catalyst may be conducted using anhydrous HF under conditions effective to convert a portion of metal oxides into corresponding metal fluorides, such as via the procedure disclosed in U.S. Pat. No. 6,780,815 to Cerri et al., the disclosure of which is expressly incorporated by reference herein. Other suitable catalysts for the dehydrofluorination reaction include metal fluorides such as chromium fluoride, alumina fluoride, iron fluoride, magnesium fluoride, and various combinations of thereof.

Other metals, such as Pd, Pt, and Ni, may also be loaded onto the above fluorinated metal oxides, for example, via a wet impregnation process wherein a salt of the metal is exposed to the fluorinated metal oxide support in solution, followed by drying and then reduction with hydrogen gas.

The amount of metal loading on the support may be from about 0.01 wt. %, about 0.05 wt. %, about 0.1 wt. %, about 0.2 wt. %, about 0.3 wt. %, about 0.4 wt. %, about 0.5 wt. %, or about 1 wt. % to about 2 wt. %, about 3 wt. %, 5 wt. % 10 wt. %, or 20 wt. %, or 30 wt. %, or 40 wt. %, or 50 wt. % or within any range encompassed by two of the foregoing values as endpoints, based on a total weight of the catalyst and support. For supported noble metal catalysts such as platinum or palladium, the metal loading may be ranged from about 0.01 wt. % to about 5 wt. %, preferably from about 0.05 wt. % to about 2 wt. %, and more preferably from about 0.1 wt. % to about 1 wt. %.

When fluorinated alumina is used, the amount of metal loading on the support may be from about 0.01 wt. % to about 5 wt. %, preferably from about 0.05 wt. % to about 2 wt. %, most preferably from about 0.1 wt. % to about 1 wt. %.

The catalyst used in step (ii) may have a proper BET (Brunauer, Emmet, and Teller) surface area. In some embodiments, the BET surface area of the catalyst may be as low as about 10 m²/g, about 20 m²/g, about 30 m²/g, about 40 m²/g, about 50 m²/g, about 60 m²/g, about 70 m²/g, about 80 m²/g, about 90 m²/g, about 100 m²/g, or as high as about 110 m²/g, about 120 m²/g, about 130 m²/g, about 140 m²/g, about 150 m²/g, about 175 m²/g, about 200 m²/g, about 225 m²/g, about 250 m²/g, about 300 m²/g, or within any range encompassed by any of the foregoing values as endpoints. For metal oxides catalysts, the BET surface area may be preferably greater than about 100 m²/g. For fluorinated metal oxides catalysts, the BET surface area may be preferably greater than about 20 m²/g. The BET analysis is the standard method for determining surface areas from nitrogen adsorption isotherms. The BET surface areas of catalysts may be measured using TriStar II Micromeritics instrument. Catalyst samples are degassed before the analysis using FlowPrep 060 instrument.

When fluorinated alumina is used, the BET surface area may be greater than about 10 m²/g, preferably greater than 20 m²/g, most preferably greater than 25 m²/g.

### Catalyst Pretreatment

The catalyst may be pretreated by drying at elevated temperatures, as low as about 200° C., about 250° C., about 300° C., about 350° C., about 360° C., about 370° C., or as high as about 380° C., about 390° C., about 400° C., about 450° C., about 500° C., about 550° C., about 600° C., or within any range encompassed by two of the foregoing values as endpoints. As part of the catalyst activation, the catalyst may be exposed to an inert gas such as N₂. The pretreatment process may take as low as about 1 hour, about 2 hours, about 3 hours, or as high about 4 hours, about 5 hours, about 6 hours, about 10 hours, about 20 hours, or within any range encompassed by two of the foregoing values as endpoints such as about 2 hours to about 4 hours, for example.

When fluorinated alumina is used, the catalyst may be pretreated by drying a temperature of from about 200° C. to about 600° C., preferably from about 300° C. to about 600° C., most preferably from about 400° C. to about 550° C.

When fluorinated alumina is used, the pretreatment process may take from about 1 hour to about 10 hours, preferably from about 2 hours to about 6 hours, most preferably from about 3 hours to about 5 hours.

### Reaction Conditions

The temperature range for dehydrofluorination reaction may be as low as about 125° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints. The temperature may be preferably from about 250° C. to about 450° C., and more preferably from about 300° C. to about 400° C.

When fluorinated alumina is used, the reaction temperature may be from about 125° C. to about 500° C., preferably from about 250° C. to about 450° C., most preferably from about 300° C. to about 400° C.

The pressure may be as little as about 1 psig, about 2 psig, about 3 psig, about 4 psig or about 5 psig, about 10 psig, about 15 psig, about 20 psig, about 25 psig, about 30 psig, about 35 psig, about 40 psig, about 50 psig, or within any range encompassed by two of the foregoing values as endpoints. For example, the pressure may be from about 1 psig to about 50 psig, preferably from about 5 psig to about 30 psig, and more preferably from about 10 psig to about 20 psig.

When fluorinated alumina is used, the reaction pressure may be from about 1 psig to about 50 psig, preferably from about 5 psig to about 30 psig, most preferably from about 10 psig to about 20 psig.

The contact time of the reactants with the catalyst may be as little as about 0.1 second, about 1 second, about 5 seconds, about 10 seconds, about 15 seconds or about 20 seconds, or as long as about 25 seconds, about 30 seconds, about 40 seconds, about 50 seconds, about 60 seconds, about 120 seconds, about or within any range encompassed by two of the foregoing values as endpoints. For example, the contact time may be from about 1 second to about 60 seconds.

When fluorinated alumina is used, the contact time may be from about 1 second to about 60 seconds, preferably from about 5 seconds to about 40 seconds, most preferably from about 10 seconds to about 30 seconds.

The reaction may also be conducted substantially in the absence of water. For example, the amount of water present during the reaction may be less than 1 mol %, less than 0.5 mol %, or less than 0.05 mol % based on a total weight of the reactants in the reactor.

### Products

In the dehydrofluorination reactions of Step (ii), the cis/trans molar ratio of the 1,2-difluoroethylene in the product mixture may be as low as about 1, about 2, about 3, about 4, about 5, about 6, or as high as about 7, about 8, about 9, about 10, about 11, about 12, about 13, about 14, about 15 or within any range encompassed by two of the foregoing values as endpoints. For example, the cis/trans ratio may be from about 2 to about 15.

When fluorinated alumina is used, the cis/trans molar ration of the 1,2-difluoroethylene in the product mixture may be from about 1 to about 15, preferably from about 1 to about 10, most preferably from about 2 to about 7.

The selectivity to the desired 1,2-difluoroethylene product (the sum of 1232E and 1232Z) may be as low as about 80%, about 85%, about 89% about 90%, about 91%, about 92%, about 93%, about 94%, about 95% or as high as about 96%, about 97%, about 98%, about 99%, or within any range encompassed by two of the foregoing values as endpoints. For example, the selectivity may be from about 89% to about 99%.

When fluorinated alumina is used, the selectivity to the desired 1,2-difluoroethylene product (the sum of 1232E and 1232Z) may be from about 85% to about 99%, preferably from about 90% to about 99%, most preferably from about 95% to about 99%.

The conversion of the starting material to 1,2-difluoroethylene may be greater than about 10%, greater than about 20%, greater than about 30%, greater than about 40%, greater than about 50%, greater than about 60%, greater than about 70%, greater than about 80%, greater than about 90%, greater than about 95%, greater than about 99%, or within any range encompassed by two of the foregoing values as endpoints.

When fluorinated alumina is used, the conversion of the starting material to 1,2-difluoroethylene may be greater than about 20%, preferably greater than about 30%, most preferably greater than about 60%.

It may also be advantageous to periodically regenerate the catalyst after prolonged use while in place in the reactor. Regeneration of the catalyst may be accomplished by any means known in the art, for example, by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C., for fluorinated catalysts or hydrogen treatment at temperatures of from about 100° C. to about 400° C., preferably from about 200° C. to about 350° C. for supported transition metal catalysts.

When fluorinated alumina is used, the catalyst may be regenerated by passing air or air diluted with nitrogen over the catalyst at temperatures of from about 100° C. to about 400° C., preferably from about 200° C. to about 375° C., for from about 0.5 hour to about 3 days. This may be followed by hydrogen fluoride treatment at temperatures of from about 100° C. to about 400° C., preferably from about 200° C. to about 350° C. Additionally, the present process advantageously avoids and/or minimizes formation of 1,1,1,-trifluoroethane (HFC-143a) wherein the products of Step (ii), including trans-1,2-difluoroethylene (HFO-1132E), may include less than 5 wt. %, less than 3 wt. %, less than 1 wt. %, less than 0.5 wt. %, or less than 0.1 wt. % of 1,1,1-trifluoroethane (HFC-143a), based on a total weight of the product composition.

When fluorinated alumina is used as a catalyst, the following properties may be present. The amount of metal loading on the support may be from about 0.01 wt. % to about 5 wt. %, preferably from about 0.05 wt. % to about 2 wt. %, most preferably from about 0.1 wt. % to about 1 wt. %. The BET surface area may be greater than about 10 m²/g, preferably greater than 20 m²/g, most preferably greater than 25 m²/g. The catalyst may be pretreated by drying a temperature of from about 200° C. to about 600° C., preferably from about 300° C. to about 600° C., most preferably from about 400° C. to about 550° C. The pretreatment process may take from about 1 hour to about 10 hours, preferably from about 2 hours to about 6 hours, most preferably from about 3 hours to about 5 hours. The reaction temperature may be from about 125° C. to about 500° C., preferably from about 250° C. to about 450° C., most preferably from about 300° C. to about 400° C. The reaction pressure may be from about 1 psig to about 50 psig, preferably from about 5 psig to about 30 psig, most preferably from about 10 psig to about 20 psig. The contact time may be from about 1 second to about 60 seconds, preferably from about 5 seconds to about 40 seconds, most preferably from about 10 seconds to about 30 seconds. The cis/trans molar ration of the 1,2-difluoroethylene in the product mixture may be from about 1 to about 15, preferably from about 1 to about 10, most preferably from about 2 to about 7. The selectivity to the desired 1,2-difluoroethylene product (the sum of 1232E and 1232Z) may be from about 85% to about 99%, preferably from about 90% to about 99%, most preferably from about 95% to about 99%. The conversion of the starting material to 1,2-difluoroethylene may be greater than about 20%, preferably greater than about 30%, most preferably greater than about 60%. The catalyst may be regenerated by passing air or air diluted with nitrogen over the catalyst at temperatures of from about 100° C. to about 400° C., preferably from about 200° C. to about 375° C., for from about 0.5 hour to about 3 days. This regeneration may be followed by hydrogen fluoride treatment at temperatures of from about 100° C. to about 400° C., preferably from about 200° C. to about 350° C.

In the above process, the amount of trans-1,2-difluoroethylene (HFO-1132E) in the product mixture may be at least 5 mol %, at least 10 mol %, or at least 20 mol %, for example, based on the total moles of organic components in the product mixture.

In the above process, the amount of cis-1,2-difluoroethylene (HFO-1132Z) in the product mixture may be at least 60 mol %, at least 80 mol %, or at least 90 mol %, for example, based on the total moles of organic components in the product mixture.

The total amount of 1,1,1-trifluoroethane (HFC-143a) in the product mixture, if present, may be greater than 0.01 mol % and less than 10 mol %, less than 5 mol %, or less than 1 mol %, for example, based on total moles of organic components in the product mixture for example from 0.01 mol % to 10 mol %, from 0.01 mol % to 5 mol %, or from 0.01 mol % to 1 mol %.

The total amount of fluoroethylene in the product mixture, if present, may be greater than 0.1 mol % and less than 10 mol %, less than 5 mol %, or less than 1 mol %, for example, based on total moles of organic components in the product mixture for example from 0.1 mol % to 10 mol %, from 0.1 mol % to 5 mol %, or from 0.1 mol % to 1 mol %.

### V. Step (iii)

### General Process

The 1,2-difluoroethylene (HFO-1132) obtained in step (ii) above may be produced as a mixture containing both the trans-1,2-difluoroethylene (HFO-1132E) and cis-1,2-difluoroethylene (HFO-1132Z) isomers.

In step (iii), the cis-1,2-difluoroethylene (HFO-1132Z) isomer may be converted to the trans-1,2-difluoroethylene (HFO-1132E) isomer either by exposure to heat and/or a catalyst to yield a final product comprising, consisting essentially of, or consisting of, the trans-1,2-difluoroethylene (HFO-1132E) isomer in high purity, such as at least about 95 wt. %, at least about 99.0 wt. %, at least about 99.9 wt. %, at least about 99.99 wt. % or greater.

The isomerization reaction may be conducted in any suitable reaction vessel or reactor, but it should preferably be constructed from materials which are resistant to corrosion such as nickel and its alloys, including Hastelloy (for example, Hastelloy C276), Inconel (for example Inconel 600), and Monel wherein the vessels which may be lined with fluoropolymers. These may be single pipe or multiple tubes packed with an isomerization catalyst

### Reaction Conditions

The temperature range for the isomerization reaction may be as low as about 100° C., about 150° C., about 200° C., about 250° C., about 300° C., about 350° C., about 400° C., about 450° C., or as high as about 500° C., about 550° C., about 600° C., about 650° C., about 700° C., about 750° C., about 800° C. or within any range encompassed by two of the foregoing values as endpoints.

The reaction may be conducted at atmospheric pressure, super-atmospheric pressure or under vacuum. The vacuum pressure can be from about 5 torr to about 760 torr. Contact time of the reactants with the catalyst may range from about 0.5 seconds to about 120 seconds, however, longer or shorter times can be used.

The reaction may also be conducted in an inert atmosphere substantially in the absence of oxygen. For example, the amount of oxygen present during the reaction may be less than 10 mol %, less than 5 mol %, or less than 1 mol % based on a total weight of the reactants in the reactor.

The reaction may also be conducted substantially in the absence of water. For example, the amount of water present during the reaction may be less than 1 mol %, less than 0.5 mol %, or less than 0.05 mol % based on a total weight of the reactants in the reactor.

As used herein, the phrase “within any range encompassing any two of these values as endpoints” literally means that any range may be selected from any two of the values listed prior to such phrase regardless of whether the values are in the lower part of the listing or in the higher part of the listing. For example, a pair of values may be selected from two lower values, two higher values, or a lower value and a higher value.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances that fall within the scope of the appended claims.

### EXAMPLES

### Examples 1-10

### Step 1—Hydrogenation of 1,1,2-trichloro-1,2,2-trifluoroethane (CFC-113) to Produce 1,1,2-trifluoroethane (HFC-143)

The following Examples 1-10 were performed according to the following procedure. A ½ inch Iconel tube reactor was cleaned and flushed, then packed with a specified amount of catalyst. The catalyst was then pretreated as set forth below and the tube reactor was heated in an oven at 250° C. for at least two hours while flowing nitrogen at atmospheric pressure to reach the foregoing temperature, followed by stopping the nitrogen flow. Once the desired temperature was reached, the reactants were flowed into the tube reactor for approximately 1 hour at the desired pressure and flow rate. This step was followed by flowing the reactants through a scrubber to remove HF and HCl. Then the reaction products were collected in a cylinder chilled with dry ice. The products were analyzed by gas chromatography (GC), and the reaction selectivity and conversion were calculated.

In Examples 1-4 and 10 below, 1 wt. % Pd on carbon was obtained by stirring activated carbon 3×5 mm pellets 100 g with a stoichiometric amount 1.7 g of palladium (II) chloride (Sigma Aldrich) together in water at 80° C. for 24 h, followed by filtration and washing using distilled water. Then, the sample was dried at 100° C., and the sample was reduced by a hydrogen flow at 250° C. for 2 h.

In Example 5 below, 1 wt. % platinum on granular carbon was obtained from Sigma-Aldrich. In Example 6, 0.5 wt. % platinum on alumina 3.2 mm pellets was obtained from Sigma-Aldrich. In Examples 7-9 Alloy 200 0.24 inch Propak catalyst, a metal packing made from Nickel 200 (99.6% nickel alloy), was obtained from Cannon Instrument company.

Table 1 below provides the reaction conditions for each Example and Table 2 provides a summary of the product mixtures.

TABLE 1
Reaction Conditions for Hydrogenation of CFC-113 to Produce HFC-143

| | | | | Reactor | Contact | | |
|---|---|---|---|---|---|---|---|
| | Feed materials and | Temp | Press. | Volume | Time | | Catalyst |
| Example | feed rates | (° C.) | (psig) | (ml) | (sec) | Catalyst | Pretreatment |
| 1 | 1,1,2-trichloro-1,2,2- | 150 | 0 | 20.14 | 24.8 | 1% Pd | catalyst was |
| | trifluoroethane, | | | | | on | dried at |
| | 1.2 ml/min, hydrogen | | | | | carbon | 250° C. for 2 |
| | rate 30 ml/min, | | | | | | hours under |
| | H₂/CFC-113 ratio | | | | | | N₂ |
| | 7.34, N₂ rate 0 | | | | | | |
| | ml/min | | | | | | |
| 2 | 1,1,2-trichloro-1,2,2- | 200 | 45 | 20.14 | 23.5 | 1% Pd | catalyst was |
| | trifluoroethane, | | | | | on | reused from |
| | 0.06 ml/min, | | | | | carbon | Ex. 1 |
| | hydrogen rate | | | | | | |
| | 80 ml/min, H₂/CFC- | | | | | | |
| | 113 ratio 6.61, N₂ | | | | | | |
| | rate 4 ml/min | | | | | | |
| 3 | 1,1,2-trichloro-1,2,2- | 200 | 90 | 20.14 | 12.7 | 1% Pd | catalyst was |
| | trifluoroethane, | | | | | on | reused form |
| | 0.1 ml/min, hydrogen | | | | | carbon | Ex. 1 |
| | rate 135 ml/min, | | | | | | |
| | H₂/CFC-113 ratio | | | | | | |
| | 6.69, N₂ rate 200 | | | | | | |
| | ml/min | | | | | | |
| 4 | 1,1,2-trichloro-1,2,2- | 200 | 45 | 20.14 | 23.0 | 1% Pd | catalyst was |
| | trifluoroethane, | | | | | on | reused from |
| | 0.06 ml/min, | | | | | carbon | Ex. 1 |
| | hydrogen rate | | | | | | |
| | 80 ml/min, H₂/CFC- | | | | | | |
| | 113 ratio 6.61, N₂ | | | | | | |
| | rate 4 ml/min | | | | | | |
| 5 | 1,1,2-trichloro-1,2,2- | 200 | 45 | 19.72 | 24.3 | 1% Pt | catalyst was |
| | trifluoroethane, | | | | | on | pretreated at |
| | 0.055 ml/min, | | | | | carbon | 250° C. for 2 |
| | hydrogen rate | | | | | | hours under |
| | 70 ml/min, H₂/CFC- | | | | | | N₂ |
| | 113 ratio 6.31, N₂ | | | | | | |
| | rate 10 ml/min | | | | | | |
| 6 | 1,1,2-trichloro-1,2,2- | 200 | 45 | 17.14 | 23.7 | 0.5% Pt | catalyst was |
| | trifluoroethane, | | | | | on | pretreated at |
| | 0.055 ml/min, | | | | | alumina | 250° C. for 2 |
| | hydrogen rate | | | | | | hours under |
| | 70 ml/min, H₂/CFC- | | | | | | N₂ |
| | 113 ratio 6.31, N₂ | | | | | | |
| | rate 0 ml/min | | | | | | |
| 7 | 1,1,2-trichloro-1,2,2- | 200 | 45 | 19.72 | 8.1 | Alloy | catalyst was |
| (Comp.) | trifluoroethane, | | | | | 200 | pretreated at |
| | 1.2 ml/min, hydrogen | | | | | | 250° C. for 2 |
| | rate 30 ml/min, | | | | | | hours under |
| | H₂/CFC-113 ratio | | | | | | N₂ |
| | 7.43, N₂ rate 0 | | | | | | |
| | ml/min | | | | | | |
| 8 | 1,1,2-trichloro-1,2,2- | 400 | 45 | 19.72 | 5.7 | Alloy | catalyst was |
| (Comp.) | trifluoroethane, | | | | | 200 | reused from |
| | 1.2 ml/min, hydrogen | | | | | | Ex. 7 |
| | rate 30 ml/min, | | | | | | |
| | H₂/CFC-113 ratio | | | | | | |
| | 7.43, N₂ rate 0 | | | | | | |
| | ml/min | | | | | | |
| 9 | 1,1,2-trichloro-1,2,2- | 500 | 45 | 19.72 | 4.9 | Alloy | catalyst was |
| (Comp.) | trifluoroethane, | | | | | 200 | reused from |
| | 1.2 ml/min, hydrogen | | | | | | Ex. 7 |
| | rate 30 ml/min, | | | | | | |
| | H₂/CFC-113 ratio | | | | | | |
| | 7.43, N₂ rate 0 | | | | | | |
| | ml/min | | | | | | |
| 10 | 1,1,2-trichloro-1,2,2- | 200 | 45 | 23.1 | 3.8 | 1% Pd | catalyst was |
| | trifluoroethane, | | | | | on | reused from |
| | 0.2 ml/min, hydrogen | | | | | carbon | Ex. 1 |
| | rate 450 ml/min, | | | | | | |
| | H₂/CFC-113 ratio | | | | | | |
| | 11.58, N₂ rate 200 | | | | | | |
| | ml/min | | | | | | |
| 11 | 1,1,2-trichloro-1,2,2- | 200 | 0 | 22.3 | 11.9 | 5% Pd | catalyst was |
| | trifluoroethane, | | | | | on | pretreated at |
| | 0.05 ml/min, | | | | | alumina | 250° C. for 2 |
| | hydrogen rate | | | | | | hours under |
| | 30 ml/min, H₂/CFC- | | | | | | N₂ |
| | 113 ratio 11.58, N₂ | | | | | | |
| | rate 30 ml/min | | | | | | |

The reactions in Table 1 provided the product mixtures in Table 2 below.

TABLE 2
Summary of Products for
Hydrogenation of CFC-113 to Produce HFC-143

| | | | Selectivity | Combined | Con- |
|---|---|---|---|---|---|
| | | | to desired | Selectivity | version |
| | | | product: | (HFC-143 | of |
| | | | 1,1,2- | and | starting |
| | Product | | trifluoro- | preferred | material |
| Ex- | Composition and | | ethane | inter- | (CFC- |
| ample | Amount (mol %) | | (HFC-143) | mediates) | 113) |
| 1 | HFC-143 (1,1,2- | 44.47 | 57.7% | 92.8% | 77.0% |
| | trifluoroethane) | | | | |
| | CFC-113 (1,1,2- | 22.97 | | | |
| | trichloro-1,2,2- | | | | |
| | trifluoroethane) | | | | |
| | HCFC-133b | 12.7 | | | |
| | (CF₂ClCH2F) | | | | |
| | HCFC-133 | 3.09 | | | |
| | (CF₂HCHFCl) | | | | |
| | HCFC-123a | 9.77 | | | |
| | (CF₂ClCHFCl | | | | |
| | isomers) | | | | |
| | HFC-152a (1,1- | 3.59 | | | |
| | difluoroethane) | | | | |
| | HFO-1123 | 2.1 | | | |
| | (trifluoroethylene) | | | | |
| | HCFC-142b (1- | 0.36 | | | |
| | chloro-1,1- | | | | |
| | difluoroethane) | | | | |
| | Unidentified | 0.95 | | | |
| | components | | | | |
| 2 | HFC-143 (1,1,2- | 68.8 | 71.6% | 90.8% | 96.0% |
| | trifluoroethane) | | | | |
| | HCFC-123a | 6.33 | | | |
| | (CF₂ClCHFCl | | | | |
| | isomers) | | | | |
| | HCFC-133b | 8.37 | | | |
| | (CF₂ClCH2F) | | | | |
| | HCFC-133 | 3.43 | | | |
| | (CF₂HCHFCl) | | | | |
| | CFC-113 (1,1,2- | 3.99 | | | |
| | trichloro-1,2,2- | | | | |
| | trifluoroethane) | | | | |
| | HFC-152a (1,1- | 6.38 | | | |
| | difluoroethane) | | | | |
| | HFO-1123 | 0.26 | | | |
| | (trifluoroethylene) | | | | |
| | HCFC-142b (1- | 0.93 | | | |
| | chloro-1,1- | | | | |
| | difluoroethane) | | | | |
| | Unidentified | 1.51 | | | |
| | components | | | | |
| 3 | HFC-143 (1,1,2- | 61.99 | 65.4% | 95.3% | 94.8% |
| | trifluoroethane) | | | | |
| | HCFC-133b | 10.76 | | | |
| | (CF₂ClCH2F) | | | | |
| | HCFC-133 | 4.88 | | | |
| | (CF₂HCHFCl) | | | | |
| | HCFC-123a | 12.1 | | | |
| | (CF₂ClCHFCl | | | | |
| | isomers) | | | | |
| | CFC-113 (1,1,2- | 5.15 | | | |
| | trichloro-1,2,2- | | | | |
| | trifluoroethane) | | | | |
| | HFC-152a (1,1- | 3.33 | | | |
| | difluoroethane) | | | | |
| | HCFC-142b (1- | 0.16 | | | |
| | chloro-1,1- | | | | |
| | difluoroethane) | | | | |
| | HFO-1123 | 0.68 | | | |
| | (trifluoroethylene) | | | | |
| | Unidentified | 0.95 | | | |
| | components | | | | |
| 4 | HFC-143 (1,1,2- | 61.85 | 57.7% | 91.7% | 89.2% |
| | trifluoroethane) | | | | |
| | CFC-113 (1,1,2- | 10.82 | | | |
| | trichloro-1,2,2- | | | | |
| | trifluoroethane) | | | | |
| | HCFC-133b | 8.26 | | | |
| | (CF₂ClCH2F) | | | | |
| | HCFC-123a | 7.39 | | | |
| | (CHFClCF₂Cl) | | | | |
| | HFC-152a (1,1- | 6.21 | | | |
| | difluoroethane) | | | | |
| | HCFC-133 | 3.74 | | | |
| | (CF₂HCHFCl) | | | | |
| | HFO-1123 | 0.55 | | | |
| | (trifluoroethylene) | | | | |
| | HCFC-142b (1- | 0.33 | | | |
| | chloro-1,1- | | | | |
| | difluoroethane) | | | | |
| | HCFC-142a | 0.28 | | | |
| | (CH₂FCHFCl) | | | | |
| | Unidentified | 0.57 | | | |
| | components | | | | |
| 5 | CFC-113 (1,1,2- | 32.14 | 28.3% | 63.2% | 67.9% |
| | trichloro-1,2,2- | | | | |
| | trifluoroethane) | | | | |
| | HFC-143 (1,1,2- | 19.25 | | | |
| | trifluoroethane) | | | | |
| | HCFC-123a | 15.48 | | | |
| | (CF₂ClCHFCl) | | | | |
| | HFC-152 (1,2- | 14.51 | | | |
| | difluoroethane) | | | | |
| | HCFC-133b | 6.45 | | | |
| | (CF₂ClCH₂F) | | | | |
| | HCFC-133 | 1.47 | | | |
| | (CF₂HCHFCl) | | | | |
| | HFC-152a (1,1- | 5.01 | | | |
| | difluoroethane) | | | | |
| | HFC-143a (1,1,1- | 2.49 | | | |
| | trifluoroethane) | | | | |
| | HFO-1123 | 0.26 | | | |
| | (trifluoroethylene) | | | | |
| | HCFC-142b (1- | 0.23 | | | |
| | chloro-1,1- | | | | |
| | difluoroethane) | | | | |
| | Unidentified | 2.71 | | | |
| | components | | | | |
| 6 | HCFC-123a | 40.94 | 19.9 | 91.4% | 74.6% |
| | (CF₂ClCHFCl | | | | |
| | isomers) | | | | |
| | CFC-113 (1,1,2- | 25.41 | | | |
| | trichloro-1,2,2- | | | | |
| | trifluoroethane) | | | | |
| | HFC-143 (1,1,2- | 14.82 | | | |
| | trifluoroethane) | | | | |
| | HCFC-133b | 10.24 | | | |
| | (CF₂ClCH₂F) | | | | |
| | HCFC-133 | 1.53 | | | |
| | (CF₂HCHFCl) | | | | |
| | HFO-1123 | 0.62 | | | |
| | (trifluoroethylene) | | | | |
| | HFC-152a (1,1- | 0.37 | | | |
| | difluoroethane) | | | | |
| | Unidentified | 6.1 | | | |
| | components | | | | |
| 7 | CFC-113 (1,1,2- | 93.41 | N/A | N/A | 6.60% |
| (Comp.) | trichloro-1,2,2- | | | | |
| | trifluoroethane) | | | | |
| | HCFC-123a | 3.99 | | | |
| | (CF₂ClCHFCl) | | | | |
| | HFC-152 (1,2- | 1.77 | | | |
| | difluoroethane) | | | | |
| | HCFC-133b | 0.23 | | | |
| | (CF₂ClCH₂F) | | | | |
| | 1,1-difluoro-1- | 0.18 | | | |
| | chloroethane | | | | |
| | Unidentified | 0.42 | | | |
| | components | | | | |
| 8 | CFC-113 (1,1,2- | 94.82 | N/A | N/A | 5.20% |
| (Comp.) | trichloro-1,2,2- | | | | |
| | trifluoroethane) | | | | |
| | HCFC-123a | 4.08 | | | |
| | (CF₂ClCHFCl | | | | |
| | isomers) | | | | |
| | HCFC-133 | 0.25 | | | |
| | (CHFClCHF₂) | | | | |
| | HCFC-133b | 0.22 | | | |
| | (CF₂ClCH₂F) | | | | |
| | CFO-113 (1,1,2- | 0.16 | | | |
| | trifluoro-2- | | | | |
| | chloroethylene) | | | | |
| | Unidentified | 0.47 | | | |
| | components | | | | |
| 9 | CFC-113 (1,1,2- | 41.54 | N/A | N/A | 58.50% |
| (Comp.) | trichloro-1,2,2- | | | | |
| | trifluoroethane) | | | | |
| | CFO-1113 (1,1,2- | 26.57 | | | |
| | trifluoro-2- | | | | |
| | chloroethylene) | | | | |
| | HCFC-133 | 15.03 | | | |
| | (CF₂HCHFCl) | | | | |
| | HCFC-123a | 6.07 | | | |
| | (CHFClCClF₂) | | | | |
| | HFO-1123 | 3.16 | | | |
| | (trifluoroethylene) | | | | |
| | CF₃CCl═CHCF₃ | 1.42 | | | |
| | HFC-152a (1,1- | 1.41 | | | |
| | difluoroethane) | | | | |
| | Unidentified | 4.8 | | | |
| | components | | | | |
| 10 | HFC-143 (1,1,2- | 42.88 | 46.4% | 92.4% | 93.0% |
| | trifluoroethane) | | | | |
| | HFO-1123 | 21.92 | | | |
| | (trifluoroethylene) | | | | |
| | HCFC-133b | 8.96 | | | |
| | (CF₂ClCH₂F) | | | | |
| | HCFC-123a | 8.34 | | | |
| | (CF₂ClCHFCl) | | | | |
| | CFC-113 (1,1,2- | 7.57 | | | |
| | trichloro-1,2,2- | | | | |
| | trifluoroethane) | | | | |
| | HFC-152a (1,1- | 4.73 | | | |
| | difluoroethane) | | | | |
| | HCFC-133 | 3.86 | | | |
| | (CHF₂CHFCl) | | | | |
| | HCFC-142b (1- | 0.38 | | | |
| | chloro-1,1- | | | | |
| | difluoroethane) | | | | |
| | Unidentified | 1.36 | | | |
| | components | | | | |
| 11 | HFC-143 (1,1,2- | 42.21 | 45.9% | 76.2% | 91.9% |
| | trifluoroethane) | | | | |
| | HCFC-133b | 20.24 | | | |
| | (CF₂ClCH2F) | | | | |
| | HCFC-123a | 6.27 | | | |
| | (CF₂ClCHFCl) | | | | |
| | CFC-113 (1,1,2- | 8.12 | | | |
| | trichloro-1,2,2- | | | | |
| | trifluoroethane) | | | | |
| | HC-160 | 2.62 | | | |
| | (chloroethane) | | | | |
| | HCFC-133 | 1.32 | | | |
| | (CHF2CHFCl) | | | | |
| | HC-150 (1,2- | 15.69 | | | |
| | dichloroethane) | | | | |
| | HCl (hydrogen | 0.28 | | | |
| | chloride) | | | | |
| | Unidentified | 3.25 | | | |
| | components | | | | |

### Example 10A: Performance of Palladium/Alumina Catalysts

Example 10A demonstrated the performance of a Pd/Al₂O₃ catalyst for the conversion of CFC-113 to R-143. The experimental apparatus used for this example is shown in FIG. 1 and described above. The apparatus included a feed system containing gas flow controllers for N₂ and H₂ and a Micromotion mass flow meter connected to a research control valve (RCV) controlling the organic flow rate. The reactor consisted of one-inch SS tube packed with the catalyst. A thermocouple was inserted into the middle of the catalyst bed to read the operating temperature. The pressure control system consisted of an RCV which was controlling the pressure by getting feedback from the pressure transducer placed after the reactor. For GC analysis, samples were taken from the reactor using a sample bag filled with 50 ml of water to capture HCl and HF. Before the GC analysis, the sample bag was heated at 60° C. for one hour to assure that all the organic content was in the vapor phase. Then, a sample was taken using a syringe and injected into the GC instrument for analysis. Table 3 shows the conversion and selectivity of a undiluted 0.2% Pd/α-Al₂O₃ catalyst with a BET surface area of 3.9 m²/g in the process of CFC-113 conversion to R-143. While the selectivity toward HFC-143 increased with temperature, the overall selectivity to HFC-143+recyclables decreased with temperature because of increased formation of undesired by-products of ethane, chlorodifluoroethane (such as HCFC-142, HCFC-142b, and HCFC-142a), HFC-143a, and HCC-160.

In Table 3 below, the product/intermediates may comprise further recyclable components such as 1-chloro-1,1,2-trifluoroethane (HCFC-133b), 1,2-dichloro-1,1,2-trifluoroethane (HCFC-123a), and trifluoroethylene (HFO-1123). These components are considered as recyclable in that they may be recycled to the input of the step (i) reaction and eventually converted to 1,1,2-trifluoroethane (HFC-143).

In Table 3 below, the by-products may comprise 1,1-difluoroethane (HFC-152a), ethane (HC-170), 1,1,1-trifluoroethane (HFC-143a), 1,1-difluoroethylene (HFO-1132a), 1-chloro-1,1-difluoroethane (HCFC-142b), chlorodifluoroethane (such as HCFC-142, HCFC-142b, and HCFC-142a), and chloroethane (HCC-160). These by-products are the result of dehydrofluorination side reactions and are difficult to recycle or convert into 1,1,2-trifluoroethane (HFC-143) and therefore may be separated from the products of the step (i) reaction for proper disposal. For the reactions in Table 3, the catalyst volume was 50 ml, the reactor pressure was 45 psig, the H2 flow rate was 150 ml/min and the HFC-113 flow rate was 10 g/h.

TABLE 3
Substrate conversion and selectivity using undiluted 0.2% Pd/α-Al₂O₃ catalyst

| Temp | Conv. | % Product/intermediates selectivity | | | | % By-product selectivity | | | | | |
|---|---|---|---|---|---|---|---|---|---|---|---|
| (° C.) | (%) | R-143 | R-133b | R-123a | total | R-152a | R-170 | R-143a | R-160 | R-142 | total |
| 150 | 90.0 | 31.5 | 51.0 | 12.4 | 95.2 | 2.6 | 0.7 | 0.3 | 0.4 | 0.2 | 4.8 |
| 175 | 99.0 | 39.0 | 43.0 | 12.0 | 94.0 | 2.0 | 2.2 | 0.3 | 1.2 | 0.3 | 6.0 |
| 200 | 99.9 | 39.5 | 36.0 | 15.0 | 90.5 | 0.2 | 3.5 | 2.0 | 2.1 | 1.7 | 9.5 |

The results in Table 3 indicate that the selectivity toward HFC-143 increased with temperature and the overall selectivity to HFC-143+recyclables decreased with temperature due to increased formation of undesired by-products such as ethane, chlorodifluoroethane, HFC-143a, and HCC-160.

### Examples 11-54

### Step 2—Dehydrofluorination of 1,1,2-trifluoroethane (HFC-143) to Produce trans-1,2-difluoroethylene (HFO-1132E) and cis-1,2-difluoroethylene (HFO-1132Z)

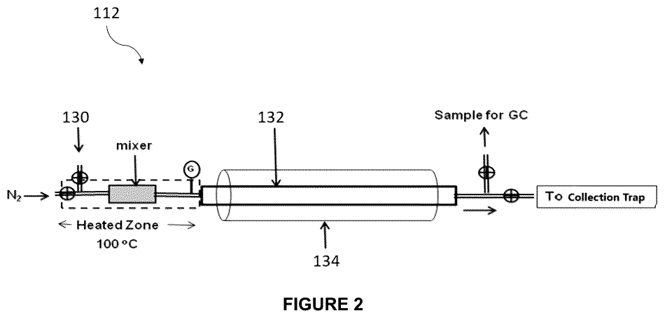

Referring to FIG. 2, the following Examples 11-54 were performed in reactor 112 according to the following procedure. A 1% inch Inconel tube reactor 132 was cleaned and flushed, then packed with a specified amount of catalyst, prepared as set forth below. The catalyst was then pretreated as set forth in Table 4 and 5 below and the tube reactor 132 was heated in an oven/furnace 134 at 250° C. for at least two hours while flowing nitrogen at atmospheric pressure to reach the foregoing temperature, followed by stopping the nitrogen flow. No oxygen or air was allowed to enter the reactor 112. Once the desired temperature was reached, the reactants were metered by a mass flow controller into the tube reactor 132 via reactant inlet 130 for approximately 1 hour at the desired pressure and flow rate. Nitrogen was metered into the tube to adjust the contact time in some cases. This step was followed by flowing the reactants through a DI water scrubber to remove HF and HCl. Aqueous 5% potassium hydroxide solution was also used to remove the HF and HCl from the reaction, no significant difference of 1132 ratio in the products was observed. Then the reaction products were collected in a cylinder chilled with dry ice followed by another cylinder chilled with liquid nitrogen. The products were analyzed by gas chromatography (GC), and the reaction selectivity and conversion was calculated.

### Preparation of Catalysts for Examples 11-54

### (I) Preparation of Fluorinated Chromium Oxide

Fluorinated chromium oxide was obtained via the procedure disclosed at col. 10, line 43 through col. 12, line 39 of U.S. Pat. No. 6,780,815 to Cerri et al. The fluorinated chromium oxide catalyst had a BET surface area of 50 m²/g. Before fluorination, the chromium oxide catalyst had a surface area of 211 m²/g.

### (II) Preparation of Fluorinated Alumina

300 g Alumina pellets 3×5 mm in a 1 inch Inconel tube reactor were slowly heated under 1 L/min N₂ to 200° C., held for 4 hours at 200° C., then ramped to 300° C. at 3° C./min heating rate. The catalyst was held for 4 hours at 300° C., then ramped to 400° C. at 3 C/min and held for 8 hours.

The catalyst was then cooled to 200° C., and N₂ was fed at 1 L/min at a pressure 20 psig, HF flow was initiated and fed into the tube reactor at ratio of 1 wt. % HF/N₂ mixture. The feeding rate was held until the catalyst temperature at all points was less than 215° C., before increasing the HF/N₂ ratio to 2.5 wt. %, 5 wt. %, 9.6 wt. %, 14 wt. %, 21 wt. %, and 25 wt. %, HF/N₂ ratio was increased to next level only after the catalyst temperature was stable or below 215° C. at all points. After reaching 25 wt. % of HF/N₂ ratio, the tube reactor temperature was slowly ramped to 350° C. at rate of 3° C./min, and this temperature was held for 2 hours while 25 wt. % HF/N₂ was continuously fed through the catalyst bed. The temperature was then ramped to 400° C. at rate of 3° C./min and held at 25 wt. % HF/N₂ for 2 hours, followed by increase of the pressure to 120 psig, and the flow of N₂ flow was stopped and switched to 100 wt. % HF at 120 psig and 1.5 lbs/h for 16 hours. The HF feed was then discontinued followed by re-starting N₂ flow at 6 L/min with cooling to room temperature.

### (III) Preparation of 1 wt. % Pd on Fluorinated Alumina (1% Pd on AlF₃)

For Examples 19-22, 1.25 g of Pd(NO₃)₂·2H₂O was dissolved in 100 ml DI water, and 50.0 g of AlF₃ pellets (5×3 mm from Johnson Matthey) were added into the solution. The resulted mixture was soaked overnight at room temperature, then water was removed under vacuum. The resulted solid was further dried at 150° C. under vacuum for 4 h to give 36.6 g pre-catalyst.

The pre-catalyst was then dried under 200 ml/min air flow at 400° C. for 4 hours, then reduced with 100 ml/min hydrogen flow at 400° C. for 2 h. The reduced catalyst was then used for the dehydrofluorination reactions below.

### (IV) Preparation of Fluorinated Magnesium Oxide

For Examples 23-26, 300 g of magnesium oxide chips (from MgO crystals) was placed in 1 inch Inconel tube and treated in the same manner as the fluorinated alumina in (II) above.

### (V) Preparation of 8.9 wt. % Ni on Fluorinated Alumina (1% Ni on AlF₃)

For Examples 27-28, 8.9% Ni on fluorinated alumina was prepared similarly, with 24.7 g of Ni(NO₃)₂·6H2O dissolved in 100 ml DI water, and 51.3 g of AlF₃ pellets was added into the solution. The resulted solution was aged overnight, then water was removed under vacuum.

The resulted solid was further dried under vacuum at 15000 for 4 h to give 51.5 g greyish solid. The pre-catalyst was further dried at 400° C. under 200 ml/min air flow for 4 hours, then reduced with 100 ml/min H₂ flow for 2 hours at 400° C. The reduced catalyst was then used for dehydrofuorination reactions below.

A summary of the reaction conditions is provided in Table 4 below

TABLE 4
Reaction Conditions for Dehydrofluorination
of HFC-143 to Produce HFO-1132E and HFO-1132Z

| | Feed | | | | | | |
|---|---|---|---|---|---|---|---|
| | materials | | | Reactor | Contact | | Catalyst |
| | and feed | Temp | Press | Volume | Time | | Pre- |
| Example | rates | (° C.) | (psia) | (ml) | (sec) | Catalyst | treatment |
| 11 | 1,1,2- | 380 | 15 | 20.57 | 6.2 | Fluorinated | catalyst |
| | trifluoroethane | | | | | chromium | was |
| | rate 80 ml/min, | | | | | oxide | slowly |
| | N₂ rate | | | | | | heated to |
| | 10 ml/min, | | | | | | 250° C., |
| | | | | | | | then at |
| | | | | | | | rate of |
| | | | | | | | 3° C./min |
| | | | | | | | to 380° C. |
| 12 | 1,1,2- | 400 | 15 | 20.57 | 3.4 | catalyst was | catalyst was |
| | trifluoroethane | | | | | reused from | reused from |
| | rate | | | | | Ex. 11 | Ex. 11 |
| | 100 ml/min, N₂ | | | | | | |
| | rate 60 ml/min, | | | | | | |
| 13 | 1,1,2- | 350 | 15 | 20.57 | 3.1 | catalyst was | catalyst was |
| | trifluoroethane | | | | | reused from | reused from |
| | rate 8 g/h, N₂ | | | | | Ex. 12 | Ex. 12 |
| | rate | | | | | | |
| | 150 ml/min, | | | | | | |
| 14 | 1,1,2- | 380 | 15 | 20.57 | 6.3 | catalyst was | catalyst was |
| | trifluoroethane | | | | | reused from | reused from |
| | rate 6 g/h, N₂ | | | | | Ex. 13 | Ex. 13 |
| | rate 60 ml/min, | | | | | | |
| 15 | 1,1,2- | 380 | 15 | 20.57 | 3.1 | catalyst was | catalyst |
| | trifluoroethane | | | | | reused from | was |
| | rate 8 g/h, N₂ | | | | | Ex. 14 | oxidized |
| | rate | | | | | | with 200 |
| | 140 ml/min, | | | | | | ml/min air |
| | | | | | | | at 250- |
| | | | | | | | 380° C. for |
| | | | | | | | 4 hours |
| | | | | | | | before |
| | | | | | | | use |
| 16 | 1,1,2- | 380 | 15 | 22.03 | 3.3 | Fluorinated | catalyst |
| | trifluoroethane | | | | | alumina | was |
| | rate 8 g/h, N₂ | | | | | (gamma- | pretreated |
| | rate | | | | | alumina | at 500° C. |
| | 140 ml/min, | | | | | pellets was | for 4 |
| | | | | | | treated with | hours |
| | | | | | | HF at 400° C., | before |
| | | | | | | 120 Psig for | use |
| | | | | | | 16 h) | |
| 17 | 1,1,2- | 350 | 15 | 22.03 | 3.3 | catalyst was | catalyst was |
| | trifluoroethane | | | | | reused from | reused from |
| | rate 8 g/h, N₂ | | | | | Ex. 16 | Ex. 16 |
| | rate | | | | | | |
| | 150 ml/min, | | | | | | |
| 18 | 1,1,2- | 380 | 15 | 22.03 | 1.1 | catalyst was | catalyst was |
| | trifluoroethane | | | | | reused from | reused from |
| | rate 8 g/h, N₂ | | | | | Ex. 17 | Ex. 17 |
| | rate | | | | | | |
| | 140 ml/min, | | | | | | |
| 19 | 1,1,2- | 380 | 15 | 22.03 | 1.1 | 1% Pd on | catalyst |
| | trifluoroethane | | | | | AlF₃ pellets | was |
| | rate 30 g/h, N₂ | | | | | (wet | pretreated |
| | rate | | | | | impregnated) | at 400° C. |
| | 400 ml/min, | | | | | | for 4 |
| | | | | | | | hours |
| | | | | | | | before |
| | | | | | | | use |
| 20 | 1,1,2- | 380 | 15 | 22.03 | 3.2 | catalyst was | catalyst was |
| | trifluoroethane | | | | | reused from | reused from |
| | rate 8 g/h, N₂ | | | | | Ex. 19 | Ex. 19 |
| | rate | | | | | | |
| | 150 ml/min, | | | | | | |
| 21 | 1,1,2- | 350 | 15 | 22.03 | 3.2 | catalyst was | catalyst was |
| | trifluoroethane | | | | | reused from | reused from |
| | rate 8 g/h, N₂ | | | | | Ex. 20 | Ex. 20 |
| | rate | | | | | | |
| | 160 ml/min, | | | | | | |
| 22 | 1,1,2- | 380 | 15 | 22.03 | 1.1 | catalyst was | catalyst was |
| | trifluoroethane | | | | | reused from | reused from |
| | rate 30 g/h, N₂ | | | | | Ex. 21 | Ex. 21 |
| | rate | | | | | | |
| | 400 ml/min, | | | | | | |
| 23 | 1,1,2- | 380 | 15 | 22.54 | 3.3 | Fluorinated | catalyst |
| | trifluoroethane | | | | | magnesium | was |
| | rate 5.5 g/h, | | | | | oxide (pellets | pretreated |
| | N₂ rate | | | | | was treated | at 400° C. |
| | 160 ml/min, | | | | | with HF at | for 4 |
| | | | | | | 400 C., | hours |
| | | | | | | 120 Psig for | before |
| | | | | | | 16 h) | use |
| 24 | 1,1,2- | 500 | 15 | 22.54 | 3.2 | catalyst was | catalyst was |
| | trifluoroethane | | | | | reused from | reused from |
| | rate 3 g/h, N₂ | | | | | Ex. 23 | Ex. 23 |
| | rate | | | | | | |
| | 145 ml/min, | | | | | | |
| 25 | 1,1,2- | 600 | 15 | 22.54 | 3.3 | catalyst was | catalyst was |
| | trifluoroethane | | | | | reused from | reused from |
| | rate 5 g/h, N₂ | | | | | Ex. 24 | Ex. 24 |
| | rate | | | | | | |
| | 115 ml/min, | | | | | | |
| 26 | 1,1,2- | 600 | 15 | 22.54 | 1.2 | catalyst was | catalyst was |
| | trifluoroethane | | | | | reused from | reused from |
| | rate 7.5 g/h, | | | | | Ex. 25 | Ex. 25 |
| | N₂ rate | | | | | | |
| | 350 ml/min, | | | | | | |
| 27 | 1,1,2- | 380 | 15 | 21.77 | 3.2 | 8.9% Ni on | catalyst |
| | trifluoroethane | | | | | AlF₃ pellets | Ni(NO₃)₂ |
| | rate 8 g/h, N₂ | | | | | (wet | on AlF₃ |
| | rate | | | | | impregnated) | (8.9% Ni) |
| | 140 ml/min, | | | | | | pellets |
| | | | | | | | was |
| | | | | | | | pretreated |
| | | | | | | | at 400° C. |
| | | | | | | | for 4 h |
| | | | | | | | with N₂, |
| | | | | | | | then |
| | | | | | | | reduced |
| | | | | | | | with |
| | | | | | | | 100% H₂ |
| | | | | | | | at 400° C. |
| | | | | | | | for 2 h |
| | | | | | | | before |
| | | | | | | | use |
| 28 | 1,1,2- | 380 | 15 | 21.77 | 1.2 | catalyst was | catalyst was |
| | trifluoroethane | | | | | reused from | reused from |
| | rate 5 g/h, N₂ | | | | | Ex. 27 | Ex. 27 |
| | rate | | | | | | |
| | 160 ml/min, | | | | | | |
| 29 | 1,1,2- | 300 | 15 | 2.57 | 3.31 | Fluorinated | catalyst |
| | trifluoroethane | | | | | alumina | was |
| | rate 5.0 g/h | | | | | (gamma- | pretreated |
| | | | | | | alumina | at 400° C. |
| | | | | | | pellets was | for 4 h |
| | | | | | | treated with | under N₂ |
| | | | | | | HF at 400° C., | flow |
| | | | | | | 120 Psig for | before |
| | | | | | | 16 h) | use |
| 30 | 1,1,2- | 320 | 15 | 2.57 | 3.26 | catalyst was | catalyst was |
| | trifluoroethane | | | | | reused from | reused from |
| | rate 4.9 g/h | | | | | Ex. 29 | Ex. 29 |
| 31 | 1,1,2- | 340 | 15 | 2.57 | 3.29 | catalyst was | catalyst was |
| | trifluoroethane | | | | | reused from | reused from |
| | rate 4.7 g/h | | | | | Ex. 30 | Ex. 30 |
| 32 | 1,1,2- | 340 | 15 | 2.57 | 3.29 | catalyst was | catalyst was |
| | trifluoroethane | | | | | reused from | reused from |
| | rate 4.7 g/h | | | | | Ex. 31 | Ex. 31 |
| 33 | 1,1,2- | 360 | 15 | 2.57 | 3.25 | catalyst was | catalyst was |
| | trifluoroethane | | | | | reused from | reused from |
| | rate 4.6 g/h | | | | | Ex. 32 | Ex. 32 |
| 34 | 1,1,2- | 360 | 15 | 2.57 | 1.25 | catalyst was | catalyst was |
| | trifluoroethane | | | | | reused from | reused from |
| | rate 12.0 g/h | | | | | Ex. 33 | Ex. 33 |
| 35 | 1,1,2- | 360 | 15 | 2.57 | 5.0 | catalyst was | catalyst was |
| | trifluoroethane | | | | | reused from | reused from |
| | rate 3.0 g/h | | | | | Ex. 34 | Ex. 34 |
| 36 | 1,1,2- | 340 | 15 | 2.57 | 4.99 | catalyst was | catalyst was |
| | trifluoroethane | | | | | reused from | reused from |
| | rate 3.1 g/h | | | | | Ex. 35 | Ex. 35 |
| 37 | 1,1,2- | 380 | 15 | 2.57 | 3.29 | catalyst was | catalyst was |
| | trifluoroethane | | | | | reused from | reused from |
| | rate 4.4 g/h | | | | | Ex. 36 | Ex. 36 |
| 38 | 1,1,2- | 380 | 15 | 2.57 | 1.21 | catalyst was | catalyst was |
| | trifluoroethane | | | | | reused from | reused from |
| | rate 12.0 g/h | | | | | Ex. 37 | Ex. 37 |
| 39 | 1,1,2- | 380 | 15 | 2.57 | 5.01 | catalyst was | catalyst was |
| | trifluoroethane | | | | | reused from | reused from |
| | rate 2.9 g/h | | | | | Ex. 38 | Ex. 38 |
| 40 | 1,1,2- | 360 | 15 | 2.57 | 3.26 | catalyst was | catalyst was |
| | trifluoroethane | | | | | reused from | reused from |
| | rate 4.6 g/h | | | | | Ex. 39 | Ex. 39 |
| 41 | 1,1,2- | 320 | 15 | 2.57 | 3.26 | Fluorinated | catalyst |
| | trifluoroethane | | | | | chromium | was |
| | rate 4.9 g/h | | | | | oxide | slowly |
| | | | | | | | heated to |
| | | | | | | | 250° C. |
| | | | | | | | under N₂, |
| | | | | | | | then at |
| | | | | | | | rate of |
| | | | | | | | 3° C./min |
| | | | | | | | to 350° C., |
| | | | | | | | and |
| | | | | | | | stayed at |
| | | | | | | | 350° C. for |
| | | | | | | | 4 h under |
| | | | | | | | N₂ flow |
| | | | | | | | before |
| | | | | | | | use |
| 42 | 1,1,2- | 320 | 15 | 2.57 | 3.26 | catalyst was | catalyst was |
| | trifluoroethane | | | | | reused from | reused from |
| | rate 4.9 g/h | | | | | Ex. 41 | Ex. 41 |
| 43 | 1,1,2- | 320 | 15 | 2.57 | 4.99 | catalyst was | catalyst was |
| | trifluoroethane | | | | | reused from | reused from |
| | rate 3.2 g/h | | | | | Ex. 42 | Ex. 42 |
| 44 | 1,1,2- | 340 | 15 | 2.57 | 3.29 | catalyst was | catalyst was |
| | trifluoroethane | | | | | reused from | reused from |
| | rate 4.7 g/h | | | | | Ex. 43 | Ex. 43 |
| 45 | 1,1,2- | 340 | 15 | 2.57 | 4.99 | catalyst was | catalyst was |
| | trifluoroethane | | | | | reused from | reused from |
| | rate 3.1 g/h | | | | | Ex. 44 | Ex. 44 |
| 46 | 1,1,2- | 360 | 15 | 2.57 | 4.99 | catalyst was | catalyst was |
| | trifluoroethane | | | | | reused from | reused from |
| | rate 3.0 g/h | | | | | Ex. 45 | Ex. 45 |
| 47 | 1,1,2- | 360 | 15 | 2.57 | 3.26 | catalyst was | catalyst was |
| | trifluoroethane | | | | | reused from | reused from |
| | rate 4.6 g/h | | | | | Ex. 46 | Ex. 46 |
| 48 | 1,1,2- | 360 | 15 | 2.57 | 1.25 | catalyst was | catalyst was |
| | trifluoroethane | | | | | reused from | reused from |
| | rate 12.0 g/h | | | | | Ex. 47 | Ex. 47 |
| 49 | 1,1,2- | 360 | 15 | 2.57 | 3.26 | Fluorinated | catalyst |
| | trifluoroethane | | | | | chromium | was |
| | rate 4.6 g/h | | | | | oxide | slowly |
| | | | | | | | heated to |
| | | | | | | | 250° C. |
| | | | | | | | under N₂, |
| | | | | | | | then at |
| | | | | | | | rate of |
| | | | | | | | 3° C./min |
| | | | | | | | to 350° C., |
| | | | | | | | and |
| | | | | | | | stayed at |
| | | | | | | | 350° C. for |
| | | | | | | | 4 h under |
| | | | | | | | N₂ flow |
| | | | | | | | before |
| | | | | | | | use |
| 50 | 1,1,2- | 360 | 15 | 2.57 | 1.25 | catalyst was | catalyst was |
| | trifluoroethane | | | | | reused from | reused from |
| | rate 12.0 g/h | | | | | Ex. 49 | Ex. 49 |
| 51 | 1,1,2- | 360 | 15 | 2.57 | 4.99 | catalyst was | catalyst was |
| | trifluoroethane | | | | | reused from | reused from |
| | rate 3.0 g/h | | | | | Ex. 50 | Ex. 50 |
| 52 | 1,1,2- | 380 | 15 | 2.57 | 3.3 | catalyst was | catalyst was |
| | trifluoroethane | | | | | reused from | reused from |
| | rate 4.4 g/h | | | | | Ex. 51 | Ex. 51 |
| 53 | 1,1,2- | 380 | 15 | 2.57 | 1.21 | catalyst was | catalyst was |
| | trifluoroethane | | | | | reused from | reused from |
| | rate 12.0 g/h | | | | | Ex. 52 | Ex. 52 |
| 54 | 1,1,2- | 380 | 15 | 2.57 | 5.01 | catalyst was | catalyst was |
| | trifluoroethane | | | | | reused from | reused from |
| | rate 2.9 g/h | | | | | Ex. 53 | Ex. 53 |

The reactions in Table 4 provided the product mixtures in Table 5 below.

TABLE 5
Summary of Products for Dehydrofluorination of HFC-143
to Produce HFO-1132E and HFO-1132Z

| | | | | Selectivity | Conversion |
|---|---|---|---|---|---|
| | | | | to desired | (HFO- |
| | | | Cis/ | product | 1132E |
| | Product | | trans | and | material |
| Ex- | Composition and | | molar | HFO- | (HFC- |
| ample | Amount (mol %) | | ratio | 1132Z) | 143) |
| 11 | HFO-1132Z (cis-1,2- | 45.58 | 2.29 | 89.0% | 73.5% |
| | difluoroethylene) | | | | |
| | HFC-143 (1,1,2- | 26.49 | | | |
| | trifluoroethane) | | | | |
| | HFO-1132E (trans- | 19.87 | | | |
| | 1,2-difluoroethylene) | | | | |
| | HFC-143a (1,1,1- | 5.97 | | | |
| | trifluoroethane) | | | | |
| | Unidentified | 5.03 | | | |
| | components | | | | |
| 12 | HFO-1132Z (cis-1,2- | 54.08 | 3.68 | 95.6% | 71.9% |
| | difluoroethylene) | | | | |
| | HFC-143 (1,1,2- | 28.06 | | | |
| | trifluoroethane) | | | | |
| | HFO-1132E (trans- | 14.71 | | | |
| | 1,2-difluoroethylene) | | | | |
| | HFC-143a (1,1,1- | 2.20 | | | |
| | trifluoroethane) | | | | |
| | Unidentified | 2.20 | | | |
| | components | | | | |
| 13 | HFC-143 (1,1,2- | 56.23 | 9.4 | 96.0% | 43.8% |
| | trifluoroethane) | | | | |
| | HFO-1132Z (cis-1,2- | 38.0 | | | |
| | difluoroethylene) | | | | |
| | HFO-1132E (trans- | 4.04 | | | |
| | 1,2-difluoroethylene) | | | | |
| | HFC-143a (1,1,1- | 0.46 | | | |
| | trifluoroethane) | | | | |
| | Unidentified | 3.54 | | | |
| | components | | | | |
| 14 | HFO-1132Z (cis-1,2- | 53.23 | 4.66 | 95.9% | 67.4% |
| | difluoroethylene) | | | | |
| | trifluoroethane) | | | | |
| | HFO-1132E (trans- | 11.43 | | | |
| | 1,2-difluoroethylene) | | | | |
| | HFC-143a (1,1,1- | 1.39 | | | |
| | trifluoroethane) | | | | |
| | Unidentified | 2.71 | | | |
| | components | | | | |
| 15 | HFC-143 (1,1,2- | 56.23 | 2.85 | 95.5% | 85.2% |
| | trifluoroethane) | | | | |
| | HFO-1132Z (cis-1,2- | 38.0 | | | |
| | difluoroethylene) | | | | |
| | HFO-1132E (trans- | 4.04 | | | |
| | 1,2-difluoroethylene) | | | | |
| | HFC-143a (1,1,1- | 0.46 | | | |
| | trifluoroethane) | | | | |
| | Unidentified | 1.63 | | | |
| | components | | | | |
| 16 | HFO-1132Z (cis-1,2- | 49.39 | 2.51 | 96.3% | 71.2% |
| | difluoroethylene) | | | | |
| | HFC-143 (1,1,2- | 28.23 | | | |
| | trifluoroethane) | | | | |
| | HFO-1132E (trans- | 19.72 | | | |
| | 1,2-difluoroethylene) | | | | |
| | HFC-143a (1,1,1- | 1.82 | | | |
| | trifluoroethane) | | | | |
| | Unidentified | 0.84 | | | |
| | components | | | | |
| 17 | HFO-1132Z (cis-1,2- | 47.52 | 3.52 | 96.9% | 62.90% |
| | difluoroethylene) | | | | |
| | HFC-143 (1,1,2- | 37.03 | | | |
| | trifluoroethane) | | | | |
| | HFO-1132E (trans- | 13.5 | | | |
| | 1,2-difluoroethylene) | | | | |
| | HFC-143a (1,1,1- | 0.91 | | | |
| | trifluoroethane) | | | | |
| | Unidentified | 1.04 | | | |
| | components | | | | |
| 18 | HFO-1132Z (cis-1,2- | 53.04 | 4.91 | 97.7% | 65.30% |
| | difluoroethylene) | | | | |
| | HFC-143 (1,1,2- | 34.66 | | | |
| | trifluoroethane) | | | | |
| | HFO-1132E (trans- | 10.80 | | | |
| | 1,2-difluoroethylene) | | | | |
| | HFC-143a (1,1,1- | 0.54 | | | |
| | trifluoroethane) | | | | |
| | Unidentified | 0.96 | | | |
| | components | | | | |
| 19 | HFO-1132Z (cis-1,2- | 52.54 | 9.38 | 97.80% | 59.50% |
| | difluoroethylene) | | | | |
| | HFC-143 (1,1,2- | 40.53 | | | |
| | trifluoroethane) | | | | |
| | HFO-1132E (trans- | 5.6 | | | |
| | 1,2-difluoroethylene) | | | | |
| | HFC-143a (1,1,1- | 0.19 | | | |
| | trifluoroethane) | | | | |
| | Unidentified | 1.14 | | | |
| | components | | | | |
| 20 | HFO-1132Z (cis-1,2- | 57.29 | 4.53 | 98.00% | 71.40% |
| | difluoroethylene) | | | | |
| | HFC-143 (1,1,2- | 28.65 | | | |
| | trifluoroethane) | | | | |
| | HFO-1132E (trans- | 12.64 | | | |
| | 1,2-difluoroethylene) | | | | |
| | HFC-143a (1,1,1- | 0.53 | | | |
| | trifluoroethane) | | | | |
| | Unidentified | 0.89 | | | |
| | components | | | | |
| 21 | HFO-1132Z (cis-1,2- | 59.56 | 4.46 | 98.2% | 74.4% |
| | difluoroethylene) | | | | |
| | HFC-143 (1,1,2- | 25.77 | | | |
| | trifluoroethane) | | | | |
| | HFO-1132E (trans- | 13.35 | | | |
| | 1,2-difluoroethylene) | | | | |
| | HFC-143a (1,1,1- | 0.55 | | | |
| | trifluoroethane) | | | | |
| | Unidentified | 0.77 | | | |
| | components | | | | |
| 22 | HFO-1132Z (cis-1,2- | 62.18 | 6.22 | 98.60% | 73.20% |
| | difluoroethylene) | | | | |
| | HFC-143 (1,1,2- | 26.81 | | | |
| | trifluoroethane) | | | | |
| | HFO-1132E (trans- | 10.00 | | | |
| | 1,2-difluoroethylene) | | | | |
| | HFC-143a (1,1,1- | 0.35 | | | |
| | trifluoroethane) | | | | |
| | Unidentified | 0.66 | | | |
| | components | | | | |
| 23 | HFC-143 (1,1,2- | 95.8 | NA | 58.30% | 4.20% |
| | trifluoroethane) | | | | |
| | HFO-1132Z (cis-1,2- | 2.3 | | | |
| | difluoroethylene) | | | | |
| | HFO-1132E (trans- | 0 | | | |
| | 1,2-difluoroethylene) | | | | |
| | HFC-143a (1,1,1- | 0 | | | |
| | trifluoroethane) | | | | |
| | Unidentified | 1.84 | | | |
| | components | | | | |
| 24 | HFC-143 (1,1,2- | 74.85 | 13.9 | 94.10% | 25.20% |
| | trifluoroethane) | | | | |
| | HFO-1132Z (cis-1,2- | 22.07 | | | |
| | difluoroethylene) | | | | |
| | HFO-1132E (trans- | 1.59 | | | |
| | 1,2-difluoroethylene) | | | | |
| | HFC-143a (1,1,1- | 0.0 | | | |
| | trifluoroethane) | | | | |
| | Unidentified | 1.49 | | | |
| | components | | | | |
| 25 | HFO-1132Z (cis-1,2- | 54.24 | 8.6 | 97.90% | 61.80% |
| | difluoroethylene) | | | | |
| | HFC-143 (1,1,2- | 38.17 | | | |
| | trifluoroethane) | | | | |
| | HFO-1132E (trans- | 6.30 | | | |
| | 1,2-difluoroethylene) | | | | |
| | HFC-143a (1,1,1- | 0.25 | | | |
| | trifluoroethane) | | | | |
| | Unidentified | 1.04 | | | |
| | components | | | | |
| 26 | HFC-143 (1,1,2- | 71.46 | 9.5 | 94.80% | 28.50% |
| | trifluoroethane) | | | | |
| | HFO-1132Z (cis-1,2- | 24.49 | | | |
| | difluoroethylene) | | | | |
| | HFO-1132E (trans- | 2.57 | | | |
| | 1,2-difluoroethylene) | | | | |
| | HFC-143a (1,1,1- | 0.20 | | | |
| | trifluoroethane) | | | | |
| | Unidentified | 1.28 | | | |
| | components | | | | |
| 27 | HFO-1132Z (cis-1,2- | 70.56 | 3.2 | 97.8% | 94.6% |
| | difluoroethylene) | | | | |
| | HFO-1132E (trans- | 21.95 | | | |
| | 1,2-difluoroethylene) | | | | |
| | HFC-143 (1,1,2- | 5.37 | | | |
| | trifluoroethane) | | | | |
| | HFC-143a (1,1,1- | 1.05 | | | |
| | trifluoroethane) | | | | |
| | Unidentified | 1.07 | | | |
| | components | | | | |
| 28 | HFO-1132Z (cis-1,2- | 73.23 | 4.5 | 98.20% | 90.90% |
| | difluoroethylene) | | | | |
| | HFO-1132E (trans- | 16.12 | | | |
| | 1,2-difluoroethylene) | | | | |
| | HFC-143 (1,1,2- | 9.05 | | | |
| | trifluoroethane) | | | | |
| | HFC-143a (1,1,1- | 0.63 | | | |
| | trifluoroethane) | | | | |
| | Unidentified | 0.44 | | | |
| | components | | | | |
| 29 | HFC-143a (1,1,1- | 0.07 | 11.52 | 94.00% | 25.31% |
| | trifluoroethane) | | | | |
| | HFO-1132E (trans- | 1.9 | | | |
| | 1,2-difluoroethylene) | | | | |
| | HFO-1132Z (cis-1,2- | 21.88 | | | |
| | difluoroethylene) | | | | |
| | HFC-143 (1,1,2- | 74.69 | | | |
| | trifluoroethane) | | | | |
| | Unidentified | 1.46 | | | |
| | components | | | | |
| 30 | HFC-143a (1,1,1- | 0.10 | 11.52 | 95.03% | 32.58% |
| | trifluoroethane) | | | | |
| | HFO-1132E (trans- | 2.48 | | | |
| | 1,2-difluoroethylene) | | | | |
| | HFO-1132Z (cis-1,2- | 28.57 | | | |
| | difluoroethylene) | | | | |
| | HFC-143 (1,1,2- | 67.42 | | | |
| | trifluoroethane) | | | | |
| | Unidentified | 1.43 | | | |
| | components | | | | |
| 31 | HFC-143a (1,1,1- | 0.06 | 15.61 | 94.89% | 28.19% |
| | trifluoroethane) | | | | |
| | HFO (fluoroethylene) | 0.03 | | | |
| | HFO-1132E (trans- | 1.61 | | | |
| | 1,2-difluoroethylene) | | | | |
| | HFO-1132Z (cis-1,2- | 25.14 | | | |
| | difluoroethylene) | | | | |
| | HFC-143 (1,1,2- | 71.81 | | | |
| | trifluoroethane) | | | | |
| | Unidentified | 1.35 | | | |
| | components | | | | |
| 32 | HFC-143a (1,1,1- | 0.10 | 13.49 | 95.90% | 34.94% |
| | trifluoroethane) | | | | |
| | HFO (fluoroethylene) | 0.04 | | | |
| | HFO-1132E (trans- | 2.31 | | | |
| | 1,2-difluoroethylene) | | | | |
| | HFO-1132Z (cis-1,2- | 31.18 | | | |
| | difluoroethylene) | | | | |
| | HFC-143 (1,1,2- | 65.06 | | | |
| | trifluoroethane) | | | | |
| | Unidentified | 1.31 | | | |
| | components | | | | |
| 33 | HFC-143a (1,1,1- | 0.117 | 12.62 | 96.20% | 33.14% |
| | trifluoroethane) | | | | |
| | HFO (fluoroethylene) | 0.02 | | | |
| | HFO-1132E (trans- | 2.73 | | | |
| | 1,2-difluoroethylene) | | | | |
| | HFO-1132Z (cis-1,2- | 34.44 | | | |
| | difluoroethylene) | | | | |
| | HFC-143 (1,1,2- | 61.29 | | | |
| | trifluoroethane) | | | | |
| | Unidentified | 1.40 | | | |
| | components | | | | |
| 34 | HFC-143a (1,1,1- | 0.03 | 13.99 | 92.80% | 19.23% |
| | trifluoroethane) | | | | |
| | HFO (fluoroethylene) | 0.03 | | | |
| | HFO-1132E (trans- | 1.19 | | | |
| | 1,2-difluoroethylene) | | | | |
| | HFO-1132Z (cis-1,2- | 16.65 | | | |
| | difluoroethylene) | | | | |
| | HFC-143 (1,1,2- | 80.77 | | | |
| | trifluoroethane) | | | | |
| | Unidentified | 1.33 | | | |
| | components | | | | |
| 35 | HFC-143a (1,1,1- | 0.11 | 14.24 | 95.90% | 40.83% |
| | trifluoroethane) | | | | |
| | HFO (fluoroethylene) | 0.05 | | | |
| | HFO-1132E (trans- | 2.57 | | | |
| | 1,2-difluoroethylene) | | | | |
| | HFO-1132Z (cis-1,2- | 36.6 | | | |
| | difluoroethylene) | | | | |
| | HFC-143 (1,1,2- | 59.17 | | | |
| | trifluoroethane) | | | | |
| | Unidentified | 1.50 | | | |
| | components | | | | |
| 36 | HFC-143a (1,1,1- | 0.03 | 15.92 | 95.30% | 21.29% |
| | trifluoroethane) | | | | |
| | HFO (fluoroethylene) | 0.06 | | | |
| | HFO-1132E (trans- | 1.2 | | | |
| | 1,2-difluoroethylene) | | | | |
| | HFO-1132Z (cis-1,2- | 19.1 | | | |
| | difluoroethylene) | | | | |
| | HFC-143 (1,1,2- | 78.71 | | | |
| | trifluoroethane) | | | | |
| | Unidentified | 1.51 | | | |
| | components | | | | |
| 37 | HFC-143a (1,1,1- | 0.17 | 11.32 | 96.90% | 48.28% |
| | trifluoroethane) | | | | |
| | HFO (fluoroethylene) | 0.08 | | | |
| | HFO-1132E (trans- | 3.8 | | | |
| | 1,2-difluoroethylene) | | | | |
| | HFO-1132Z (cis-1,2- | 43.0 | | | |
| | difluoroethylene) | | | | |
| | HFC-143 (1,1,2- | 51.72 | | | |
| | trifluoroethane) | | | | |
| | Unidentified | 1.23 | | | |
| | components | | | | |
| 38 | HFC-143a (1,1,1- | 0.04 | 14.9 | 92.90% | 22.24% |
| | trifluoroethane)ea | | | | |
| | HFO (fluoroethylene) | 0.03 | | | |
| | HFO-1132E (trans- | 1.2 | | | |
| | 1,2-difluoroethylene) | | | | |
| | HFO-1132Z (cis-1,2- | 19.37 | | | |
| | difluoroethylene) | | | | |
| | HFC-143 (1,1,2- | 77.76 | | | |
| | trifluoroethane) | | | | |
| | Unidentified | 1.50 | | | |
| | components | | | | |
| 39 | HFC-143a (1,1,1- | 0.19 | 11.66 | 96.70% | 50.51% |
| | trifluoroethane) | | | | |
| | HFO (fluoroethylene) | 0.08 | | | |
| | HFO-1132E (trans- | 3.86 | | | |
| | 1,2-difluoroethylene) | | | | |
| | HFO-1132Z (cis-1,2- | 44.99 | | | |
| | difluoroethylene) | | | | |
| | HFC-143 (1,1,2- | 49.49 | | | |
| | trifluoroethane) | | | | |
| | Unidentified | 1.39 | | | |
| | components | | | | |
| 40 | HFC-143a (1,1,1- | 0.14 | 11.47 | 96.95% | 41.94% |
| | trifluoroethane) | | | | |
| | HFO (fluoroethylene) | 0.06 | | | |
| | HFO-1132E (trans- | 3.26 | | | |
| | 1,2-difluoroethylene) | | | | |
| | HFO-1132Z (cis-1,2- | 37.40 | | | |
| | difluoroethylene) | | | | |
| | HFC-143 (1,1,2- | 58.06 | | | |
| | trifluoroethane) | | | | |
| | Unidentified | 1.80 | | | |
| | components | | | | |
| 41 | HFC-143a (1,1,1- | 0.95 | 6.38 | 94.40% | 41.70% |
| | trifluoroethane), | | | | |
| | HFO (fluoroethylene) | 0.07 | | | |
| | HFO-1132E (trans- | 5.33 | | | |
| | 1,2-difluoroethylene) | | | | |
| | HFO-1132Z (cis-1,2- | 34.03 | | | |
| | difluoroethylene) | | | | |
| | HFC-143 (1,1,2- | 58.30 | | | |
| | trifluoroethane) | | | | |
| | Unidentified | 1.32 | | | |
| | components | | | | |
| 42 | HFC-143a (1,1,1- | 0.50 | 8.04 | 94.80% | 35.08% |
| | trifluoroethane) | | | | |
| | HFO-1132E (trans- | 3.68 | | | |
| | 1,2-difluoroethylene) | | | | |
| | HFO-1132Z (cis-1,2- | 29.57 | | | |
| | difluoroethylene) | | | | |
| | HFC-143 (1,1,2- | 64.92 | | | |
| | trifluoroethane) | | | | |
| | Unidentified | 1.33 | | | |
| | components | | | | |
| 43 | HFC-143a (1,1,1- | 0.43 | 8.7 | 94.90% | 34.82% |
| | trifluoroethane) | | | | |
| | HFO-1132E (trans- | 3.41 | | | |
| | 1,2-difluoroethylene) | | | | |
| | HFO-1132Z (cis-1,2- | 29.65 | | | |
| | difluoroethylene) | | | | |
| | HFC-143 (1,1,2- | 65.18 | | | |
| | trifluoroethane) | | | | |
| | Unidentified | 1.40 | | | |
| | components | | | | |
| 44 | HFC-143a (1,1,1- | 0.47 | 7 | 95.50% | 37.25% |
| | trifluoroethane) | | | | |
| | HFO (fluoroethylene) | 0.03 | | | |
| | HFO-1132E (trans- | 4.45 | | | |
| | 1,2-difluoroethylene) | | | | |
| | HFO-1132Z (cis-1,2- | 31.14 | | | |
| | difluoroethylene) | | | | |
| | HFC-143 (1,1,2- | 62.75 | | | |
| | trifluoroethane) | | | | |
| | Unidentified | 1.16 | | | |
| | components | | | | |
| 45 | HFC-143a (1,1,1- | 0.33 | 8.73 | 95.40% | 34.80% |
| | trifluoroethane) | | | | |
| | HFO (fluoroethylene) | 0.03 | | | |
| | HFO-1132E (trans- | 3.41 | | | |
| | 1,2-difluoroethylene) | | | | |
| | HFO-1132Z (cis-1,2- | 28.79 | | | |
| | difluoroethylene) | | | | |
| | HFC-143 (1,1,2- | 65.20 | | | |
| | trifluoroethane) | | | | |
| | Unidentified | 1.25 | | | |
| | components | | | | |
| 46 | HFC-143a (1,1,1- | 0.55 | 6.23 | 96.10% | 44.58% |
| | trifluoroethane) | | | | |
| | HFO (fluoroethylene) | 0.06 | | | |
| | HFO-1132E (trans- | 5.93 | | | |
| | 1,2-difluoroethylene) | | | | |
| | HFO-1132Z (cis-1,2- | 36.92 | | | |
| | difluoroethylene) | | | | |
| | HFC-143 (1,1,2- | 55.42 | | | |
| | trifluoroethane) | | | | |
| | Unidentified | 1.12 | | | |
| | components | | | | |
| 47 | HFC-143a (1,1,1- | 0.18 | 9.09 | 93.50% | 25.16% |
| | trifluoroethane) | | | | |
| | HFO (fluoroethylene) | 0.03 | | | |
| | HFO-1132E (trans- | 2.33 | | | |
| | 1,2-difluoroethylene) | | | | |
| | HFO-1132Z (cis-1,2- | 21.19 | | | |
| | difluoroethylene) | | | | |
| | HFC-143 (1,1,2- | 74.84 | | | |
| | trifluoroethane) | | | | |
| | Unidentified | 1.43 | | | |
| | components | | | | |
| 48 | HFC-143a (1,1,1- | 0.06 | 9.33 | 87.40% | 11.93% |
| | trifluoroethane) | | | | |
| | HFO (fluoroethylene) | 0.02 | | | |
| | HFO-1132E (trans- | 1.01 | | | |
| | 1,2-difluoroethylene) | | | | |
| | HFO-1132Z (cis-1,2- | 9.42 | | | |
| | difluoroethylene) | | | | |
| | HFC-143 (1,1,2- | 88.07 | | | |
| | trifluoroethane) | | | | |
| | Unidentified | 1.42 | | | |
| | components | | | | |
| 49 | HFC-143a (1,1,1- | 2.38 | 4.41 | 93.30% | 58.61% |
| | trifluoroethane) | | | | (70% |
| | HFO-1132E (trans- | 10.11 | | | material |
| | 1,2-diHFO | | | | balance) |
| | (fluoroethylene)) | | | | |
| | HFO-1132Z (cis-1,2- | 44.59 | | | |
| | difluoroethylene) | | | | |
| | HFC-143 (1,1,2- | 41.39 | | | |
| | trifluoroethane) | | | | |
| | Unidentified | 1.53 | | | |
| | components | | | | |
| 50 | HFC-143a (1,1,1- | 0.27 | 7.94 | 91.40% | 21.92% |
| | trifluoroethane) | | | | (75% |
| | HFO-1132E (trans- | 2.24 | | | material |
| | 1,2-difluoroethylene) | | | | balance) |
| | HFO-1132Z (cis-1,2- | 17.79 | | | |
| | difluoroethylene) | | | | |
| | HFC-143 (1,1,2- | 78.08 | | | |
| | trifluoroethane) | | | | |
| | Unidentified | 1.62 | | | |
| | components | | | | |
| 51 | HFC-143a (1,1,1- | 1.73 | 3.97 | 95.90% | 67.90% |
| | trifluoroethane) | | | | (73% |
| | HFO (fluoroethylene) | 0.07 | | | material |
| | HFO-1132E (trans- | 13.11 | | | balance) |
| | 1,2-difluoroethylene) | | | | |
| | HFO-1132Z (cis-1,2- | 52.03 | | | |
| | difluoroethylene) | | | | |
| | HFC-143 (1,1,2- | 32.08 | | | |
| | trifluoroethane) | | | | |
| | Unidentified | 0.98 | | | |
| | components | | | | |
| 52 | HFC-143a (1,1,1- | 0.53 | 6.64 | 94.90% | 36.46% |
| | trifluoroethane) | | | | (91% |
| | HFO-1132E (trans- | 4.53 | | | material |
| | 1,2-difluoroethylene) | | | | balance) |
| | HFO-1132Z (cis-1,2- | 30.08 | | | |
| | difluoroethylene) | | | | |
| | HFC-143 (1,1,2- | 63.54 | | | |
| | trifluoroethane) | | | | |
| | Unidentified | 1.32 | | | |
| | components | | | | |
| 53 | HFC-143a (1,1,1- | 0.42 | 6.1 | 93.80% | 26.94% |
| | trifluoroethane) | | | | (91% |
| | HFO-1132E (trans- | 3.56 | | | material |
| | 1,2-difluoroethylene) | | | | balance) |
| | HFO-1132Z (cis-1,2- | 21.71 | | | |
| | difluoroethylene) | | | | |
| | HFC-143 (1,1,2- | 73.06 | | | |
| | trifluoroethane) | | | | |
| | Unidentified | 1.25 | | | |
| | components | | | | |
| 54 | HFC-143a (1,1,1- | 0.53 | 7.19 | 95.40% | 40.77% |
| | trifluoroethane) | | | | (73% |
| | HFO-1132E (trans- | 4.75 | | | material |
| | 1,2-difluoroethylene) | | | | balance) |
| | HFO-1132Z (cis-1,2- | 34.16 | | | |
| | difluoroethylene) | | | | |
| | HFC-143 (1,1,2- | 59.23 | | | |
| | trifluoroethane) | | | | |
| | Unidentified | 1.33 | | | |
| | components | | | | |

### Examples 55-62

### Hydrogenation of 1,1,2-trichloro-1,2,2-trifluoroethane (CFC-113) to Produce 1,1,2-trifluoroethane (HFC-143)

Examples 1-5 and 10 above were repeated with different temperatures, pressures, contact times, and reactant ratios were tried, and the results are summarized in Table 6 below.

TABLE 6
Summary of Conditions for Examples 55-62

| | | Pres- | Contact | H2/CFC- | HFC-143 | CFC-113 |
|---|---|---|---|---|---|---|
| Exam- | Temp/ | sure | Time | 113 | Selectivity | Conversion |
| ple | ° C. | (psig) | (sec) | Ratio | (%) | (%) |
| 55 | 150 | 45 | 23.4 | 6.38 | 70.0 | 74.5 |
| 56 | 200 | 45 | 23.5 | 6.61 | 80.0 | 89.2 |
| 57 | 250 | 45 | 21.3 | 6.61 | 80.1 | 96 |
| 58 | 200 | 45 | 13.0 | 6.38 | 80.8 | 95.7 |
| 59 | 200 | 90 | 12.7 | 6.69 | 80.3 | 94.9 |
| 60 | 150 | 0 | 22.2 | 7.34 | 72.3 | 88.2 |
| 61 | 200 | 0 | 22.2 | 7.34 | 72.7 | 83.3 |
| 62 | 200 | 0 | 12.2 | 6.94 | 69.4 | 87.0 |

### Aspects

Aspect 1 is a method for producing 1,1,2-trifluoroethane (HFC-143), comprising: hydrogenating 1,1,2-trichloro-1,2,2-trifluoroethane (CFC-113) by reaction with hydrogen in the presence of a catalyst to produce 1,1,2-trifluoroethane (HFC-143).

Aspect 2 is the method of Aspect 1, wherein the catalyst is selected from the group consisting of palladium metal and platinum metal.

Aspect 3 is the method of Aspect 1 or Aspect 2, wherein the catalyst is supported on a support selected from the group consisting of carbon and alumina.

Aspect 4 is the method of any one of Aspects 1-3, wherein the catalyst is palladium supported on a carbon support.

Aspect 5 is the method of any one of Aspects 1-4, wherein the catalyst is palladium supported on an alpha alumina support.

Aspect 6 is the method of any one of Aspects 1-5, wherein the catalyst is platinum supported on a carbon support.

Aspect 7 is the method of any one of Aspects 1-6, wherein the catalyst is platinum supported on an alumina support.

Aspect 8 is the method of Aspect 7, wherein the support is selected from alpha alumina, gamma alumina, delta alumina, and theta alumina.

Aspect 9 is the method of any one of Aspects 1-8, wherein the hydrogenation step is carried out at a temperature from about 100° C. to about 600° C.

Aspect 10 is the method of any one of Aspects 1-9, wherein the hydrogenation step is carried out at a temperature from about 100° C. to about 300° C.

Aspect 11 is the method of any one of Aspects 1-10, wherein the hydrogenation step is carried out at a temperature from about 150° C. to about 250° C.

Aspect 12 is the method of any one of Aspects 1-11, wherein the hydrogenation step is carried out at a pressure of from about 10 psig to about 100 psig.

Aspect 13 is the method of any one of Aspects 1-12, wherein the hydrogenation step is carried out at a mole ratio of hydrogen to 1,1,2-trichloro-1,2,2-trifluoroethane (CFC-113) of from about 4:1 to about 10:1.

Aspect 14 is the method of any one of Aspects 1-13, wherein the hydrogenation step achieves a selectivity to 1,1,2-trifluoroethane (HFC-143) of at least 30%.

Aspect 15 is the method of any one of Aspects 1-14, wherein the hydrogenation step achieves a conversion of 1,1,2-trichloro-1,2,2-trifluoroethane (CFC-113) to 1,1,2-trifluoroethane (HFC-143) of at least 30%.

Aspect 16 is the method of any one of Aspects 1-15, wherein the hydrogenation step is carried out at a contact time of from about 1 second to about 60 seconds.

Aspect 17 is the method of any one of Aspects 1-16, further comprising the additional step of: dehydrofluorinating 1,1,2-trifluoroethane (HFC-143) in the presence of a catalyst to produce trans-1,2-difluoroethylene (HFO-1132E) and/or cis-1,2-difluoroethylene (HFO-1132Z).

Aspect 18 is the method of Aspect 17, wherein the catalyst is selected from the group consisting of a fluorinated chromium oxide, a fluorinated aluminum oxide, a fluorinated iron oxide, fluorinated magnesium oxide, a chromium fluoride, an alumina fluoride, an iron fluoride, and a magnesium fluoride.

Aspect 19 is the method of Aspect 17 or Aspect 18, wherein the catalyst further comprises at least one of palladium and nickel loaded onto a support selected from a fluorinated chromium oxide, a fluorinated aluminum oxide, and a fluorinated magnesium oxide.

Aspect 20 is the method of Aspect 17, wherein the catalyst is fluorinated chromium oxide.

Aspect 21 is the method of Aspect 17, wherein the catalyst is fluorinated aluminum oxide.

Aspect 22 is the method of Aspect 17, wherein the catalyst is fluorinated magnesium oxide.

Aspect 23 is the method of Aspect 17, wherein the catalyst is palladium supported on fluorinated aluminum oxide.

Aspect 24 is the method of Aspect 17, wherein the catalyst is nickel supported on fluorinated aluminum oxide.

Aspect 25 is the method of any one of Aspects 17-24, wherein the dehydrofluorination step is carried out at a temperature from about 200° C. to about 600° C.

Aspect 26 is the method of any one of claims 17-24, wherein the dehydrofluorination step is carried out at least one of the following conditions: (i) a temperature from about 250° C. to about 450° C.; or (ii) a pressure of from about 1 psig to about 50 psig.

Aspect 27 is the method of any one of claims 17-24, wherein the dehydrofluorination step is carried out at least one of the following conditions: (i) in an inert atmosphere wherein the amount of oxygen is less than 0.05 wt. % based on a total weight of the reactants in the reactor; or (ii) in an inert atmosphere wherein the amount of water is less than 0.05 wt. %.

Aspect 28 is the method of any one of Aspects 17-27, wherein the cis/trans molar ratio of the cis-1,2-difluoroethylene (HFO-1132Z) to trans-1,2-difluoroethylene (HFO-1132E) products is from 2 to 15.

Aspect 29 is the method of any one of Aspects 17-28, wherein the dehydrofluorination step achieves a selectivity to trans-1,2-difluoroethylene (HFO-1132E) and cis-1,2-difluoroethylene (HFO-1132Z) of from about 80% to about 99%.

Aspect 30 is the method of any one of Aspects 17-29, wherein the dehydrofluorination step achieves a conversion of 1,1,2-trifluoroethane (HFC-143) to trans-1,2-difluoroethylene (HFO-1132E) and cis-1,2-difluoroethylene (HFO-1132Z) of from about 50% to about 99%.

Aspect 31 is the method of any one of Aspects 17-30, wherein the dehydrofluorination step is carried out at a contact time of from about 1 second to about 60 seconds.

Aspect 32 is the method of any of Aspects 17-31, further comprising the additional step of: isomerizing cis-1,2-difluoroethylene (HFO-1132Z) to produce trans-1,2-difluoroethylene (HFO-1132E).

Aspect 33 is a composition produced from the method of any one of Aspects 17-32, comprising: trans-1,2-difluoroethylene (HFO-1132E) present in an amount of at least 95 wt. %; and 1,1,1-tritfluoroethane (HFC-143a) present in an amount of less than 5 wt. %, based on a total weight of the composition.

Aspect 34 is the composition of Aspect 33, comprising: trans-1,2-difluoroethylene (HFO-1132E) present in an amount of at least 97 wt. %; and 1,1,1-tritfluoroethane (HFC-143a) present in an amount of less than 3 wt. %, based on a total weight of the composition.

Aspect 35 is the composition of Aspect 34, comprising: trans-1,2-difluoroethylene (HFO-1132E) present in an amount of at least 97 wt. %; and 1,1,1-tritfluoroethane (HFC-143a) present in an amount of less than 3 wt. %, based on a total weight of the composition.

Aspect 36 is a method for producing trans-1,2-difluoroethylene (HFO-1132E), comprising: hydrogenating 1,1,2-trichloro-1,2,2-trifluoroethane (CFC-113) by reaction with hydrogen in the presence of a catalyst to produce 1,1,2-trifluoroethane (HFC-143); and dehydrofluorinating 1,1,2-trifluoroethane (HFC-143) in the presence of a catalyst to produce trans-1,2-difluoroethylene (HFO-1132E) and/or cis-1,2-difluoroethylene (HFO-1132Z).

Aspect 37 is the method of Aspect 36, further comprising the additional step of: isomerizing cis-1,2-difluoroethylene (HFO-1132Z) to produce trans-1,2-difluoroethylene (HFO-1132E).

Aspect 38 is a method for producing 1,1,2-trifluoroethane (HFC-143), comprising: hydrogenating 1,1,2-trichloro-1,2,2-trifluoroethane (CFC-113) by reaction with hydrogen in the presence of a catalyst comprising palladium or platinum to produce a product mixture, wherein the product mixture comprises: at least 30 mol % of 1,1,2-trifluoroethane (HFC-143); and 0.1 mol % to 60 mol % of at least one of 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123), based on total moles of organic components in the product mixture.

Aspect 39 is the method of Aspect 38, wherein the product mixture further comprises at least one of: 0.1 mol % to 10 mol % of 1,1-difluoroethane (HFC-152a); 0.1 mol % to 5 mol % of ethane (HC-170); 0.1 mol % to 3 mol % of chloroethane (HCC-160); 0.1 mol % to 3 mol % of 1-chloro-1,2-difluoroethane (HCFC-142a); and 0.1 mol % to 3 mol % of 1,1,1-trifluoroethane (HFC-143a), based on total moles of organic components in the product mixture.

Aspect 40 is the method of either Aspect 38 or Aspect 39, wherein the catalyst is supported on a support selected from the group consisting of carbon and alumina.

Aspect 41 is the method of any one of Aspects 38-40, wherein the hydrogenation step is carried out at a temperature from about 150° C. to about 250° C.

Aspect 42 is the method of any one of Aspects 38-41, wherein the hydrogenation step is carried out at a pressure of from about 10 psig to about 100 psig.

Aspect 43 is the method of any one of Aspects 38-42, wherein the hydrogenation step is carried out at a mole ratio of hydrogen to 1,1,2-trichloro-1,2,2-trifluoroethane (CFC-113) from about 4:1 to about 10:1.

Aspect 44 is the method of any one of Aspects 38-43, wherein the hydrogenation step achieves a selectivity to 1,1,2-trifluoroethane (HFC-143) of at least 30%.

Aspect 45 is the method of any one of Aspects 38-44, wherein the hydrogenation step achieves a selectivity to 1,1,2-trifluoroethane (HFC-143) of at least 70%.

Aspect 46 is the method of any one of Aspects 38-45, wherein the hydrogenation step achieves a combined selectivity to 1,1,2-trifluoroethane (HFC-143), 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) of greater than 75%.

Aspect 47 is the method of any one of Aspects 38-46, wherein the hydrogenation step achieves a combined selectivity to 1,1,2-trifluoroethane (HFC-143), 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) of greater than 90%.

Aspect 48 is the method of any one of Aspects 38-47, wherein the hydrogenation step achieves a conversion of 1,1,2-trichloro-1,2,2-trifluoroethane (CFC-113) to 1,1,2-trifluoroethane (HFC-143) of at least 75%.

Aspect 49 is the method of any one of Aspects 38-48, wherein the hydrogenation step achieves a conversion of 1,1,2-trichloro-1,2,2-trifluoroethane (CFC-113) to 1,1,2-trifluoroethane (HFC-143) of at least 90%.

Aspect 50 is the method of any one of Aspects 38-49, wherein the hydrogenation step achieves a selectivity to 1,1-difluoroethane (HFC-152a), ethane (HC-170), chloroethane (HCC-160), 1,1,1-trifluoroethane (HFC-143a), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)), and of less than 10%.

Aspect 51 is the method of any one of Aspects 38-50, wherein the hydrogenation step is carried out at a contact time of from about 5 second to about 40 seconds.

Aspect 52 is a method for producing trans-1,2-difluoroethylene (HFO-1132E), comprising: dehydrofluorinating 1,1,2-trifluoroethane (HFC-143) in the presence of a catalyst selected from fluorinated alumina, palladium supported on fluorinated alumina, fluorinated magnesium oxide, and nickel supported on fluorinated alumina, to produce a product mixture comprising trans-1,2-difluoroethylene (HFO-1132E) and/or cis-1,2-difluoroethylene (HFO-1132Z).

Aspect 53 is the method of Aspect 52, wherein the catalyst is fluorinated alumina.

Aspect 54 is the method of either Aspect 52 or Aspect 53, wherein the reactant mixture comprises a cis/trans molar ratio of the cis-1,2-difluoroethylene (HFO-1132Z) to trans-1,2-difluoroethylene (HFO-1132E) from 2 to 15.

Aspect 55 is the method of any one of Aspects 52-54, wherein the dehydrofluorination step achieves a selectivity to trans-1,2-difluoroethylene (HFO-1132E) and cis-1,2-difluoroethylene (HFO-1132Z) of greater than about 80%.

Aspect 56 is the method of any one of Aspects 52-55, wherein the products of the dehydrofluorination step includes less than 1 mol % of HFC-143a, based on total moles of organic components in the product mixture.

Aspect 57 is the method of any one of Aspects 52-56, wherein the dehydrofluorination step is carried out comprising at least one of the following conditions: (i) a temperature from about 300° C. to about 400° C.; (ii) a pressure of from about 5 psig to about 30 psig; (iii) at a contact time of from about 1 second to about 60 seconds; (iv) an inert atmosphere wherein an amount of oxygen present is less than 0.05 wt. % based on a total weight of reactants in a reactor; and (v) in an inert atmosphere wherein an amount of water present is less than 0.05 wt. % based on a total weight of reactants in a reactor.

What is claimed is:

1. A method for producing 1,1,2-trifluoroethane (HFC-143), comprising:
hydrogenating 1,1,2-trichloro-1,2,2-trifluoroethane (CFC-113) by reaction with hydrogen in the presence of a catalyst comprising palladium or platinum to produce a product mixture, wherein the product mixture comprises:
at least 30 mol % of 1,1,2-trifluoroethane (HFC-143); and
0.1 mol % to 60 mol % of at least one of 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123), based on total moles of organic components in the product mixture.

2. The method of claim 1, wherein the product mixture further comprises at least one of:
0.1 mol % to 10 mol % of 1,1-difluoroethane (HFC-152a);
0.1 mol % to 5 mol % of ethane (HC-170);
0.1 mol % to 3 mol % of chloroethane (HCC-160);
0.1 mol % to 3 mol % of 1-chloro-1,2-difluoroethane (HCFC-142a); and
0.1 mol % to 3 mol % of 1,1,1-trifluoroethane (HFC-143a), based on total moles of organic components in the product mixture.

3. The method of claim 1 wherein the catalyst is supported on a support selected from the group consisting of carbon and alumina.

4. The method of claim 1, wherein the hydrogenation step is carried out at a temperature from about 150° C. to about 250° C.

5. The method of claim 1, wherein the hydrogenation step is carried out at a pressure of from about 10 psig to about 100 psig.

6. The method of claim 1, wherein the hydrogenation step is carried out at a mole ratio of hydrogen to 1,1,2-trichloro-1,2,2-trifluoroethane (CFC-113) from about 4:1 to about 10:1.

7. The method of claim 1, wherein the hydrogenation step achieves a selectivity to 1,1,2-trifluoroethane (HFC-143) of at least 30%.

8. The method of claim 1, wherein the hydrogenation step achieves a selectivity to 1,1,2-trifluoroethane (HFC-143) of at least 70%.

9. The method of claim 1, wherein the hydrogenation step achieves a combined selectivity to 1,1,2-trifluoroethane (HFC-143), 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) of greater than 75%.

10. The method of claim 1, wherein the hydrogenation step achieves a combined selectivity to 1,1,2-trifluoroethane (HFC-143), 1-chloro-1,1,2-trifluoroethane (HCFC-133b), 1-chloro-1,2,2-trifluoroethane (HCFC-133), 1,2-dichloro-1,1,2-trifluoroethane (HCFC-123a), and trifluoroethylene (HFO-1123) of greater than 90%.

11. The method of claim 1, wherein the hydrogenation step achieves a conversion of 1,1,2-trichloro-1,2,2-trifluoroethane (CFC-113) to 1,1,2-trifluoroethane (HFC-143) of at least 75%.

12. The method of claim 1, wherein the hydrogenation step achieves a conversion of 1,1,2-trichloro-1,2,2-trifluoroethane (CFC-113) to 1,1,2-trifluoroethane (HFC-143) of at least 90%.

13. The method of claim 1, wherein the hydrogenation step achieves a selectivity to 1,1-difluoroethane (HFC-152a), ethane (HC-170), chloroethane (HCC-160), 1,1,1-trifluoroethane (HFC-143a), chlorodifluoroethane (such as 1-chloro-2,2-difluoroethane (HCFC-142), 1-chloro-1,1-difluoroethane (HCFC-142b), and 1-chloro-1,2-difluoroethane (HCFC-142a)) of less than 10%.

14. The method of claim 1, wherein the hydrogenation step is carried out at a contact time of from about 5 second to about 40 seconds.

15. A method for producing trans-1,2-difluoroethylene (HFO-1132E), comprising:
dehydrofluorinating 1,1,2-trifluoroethane (HFC-143) in the presence of a catalyst selected from fluorinated alumina, palladium supported on fluorinated alumina, fluorinated magnesium oxide, and nickel supported on fluorinated alumina, to produce a product mixture comprising trans-1,2-difluoroethylene (HFO-1132E) and/or cis-1,2-difluoroethylene (HFO-1132Z).

16. The method of claim 15, wherein the catalyst is fluorinated alumina.

17. The method of claim 15, wherein the product mixture comprises a cis/trans molar ratio of the cis-1,2-difluoroethylene (HFO-1132Z) to trans-1,2-difluoroethylene (HFO-1132E) from 2 to 15.

18. The method of claim 15, wherein the dehydrofluorination step achieves a selectivity to trans-1,2-difluoroethylene (HFO-1132E) and cis-1,2-difluoroethylene (HFO-1132Z) of greater than about 80%.

19. The method of claim 15, wherein the product mixture includes less than 1 mol % of HFC-143a, based on total moles of organic components in the product mixture.

20. The method of claim 15, wherein the dehydrofluorination step is carried out comprising at least one of the following conditions:
(i) a temperature from about 300° C. to about 400° C.;
(ii) a pressure of from about 5 psig to about 30 psig;
(iii) a contact time of from about 1 second to about 60 seconds;
(iv) an inert atmosphere wherein an amount of oxygen present is less than 0.05 wt. % based on a total weight of reactants in a reactor; and
(v) in an inert atmosphere wherein an amount of water present is less than 0.05 wt. % based on a total weight of reactants in a reactor.
